(19) United States
(12) Patent Application Publication
(10) Pub. No.: US 2011/0177161 A1
(43) Pub. Date: Jul. 21, 2011

(54) PHARMACEUTICAL COMPOSITIONS OF [5(S)-(2'-HYDROXYETHOXY)-20(S)-CAMPTOTHECIN

(75) Inventors: Vijay Kumar Nekkanti, Narasapur (IN); Pradeep Jairao Karatgi, Hyderabad (IN); Mahesh Paithankar, Kopargaon (IN); Raviraj Sukumar Pillai, Hyderabad (IN); Akella Venkateswarlu, Hyderabad (IN); Alikunju Shanvas, Secunderabad (IN); Reka Ajay Kumar, Hyderabad (IN); Mullangi Ramesh, Hyderabad (IN); Sirisilla Raju, Hyderabad (IN); Duvvuri Subrahmanyam, Hyderabad (IN); Rajagopal Sriram, Chennai (IN)

(73) Assignees: DR. REDDY'S LABORATORIES LIMITED, Hyderabad 500 016, Andhra Pradesh (IN); DR. REDDY'S LABORATORIES, INC., Bridgewater, NJ (US)

(21) Appl. No.: 12/601,357

(22) PCT Filed: May 27, 2008
(86) PCT No.: PCT/US08/64841

§ 371 Date: Sep. 10, 2010

(30) Foreign Application Priority Data
May 24, 2007 (IN) 1090/CHE/2007
Nov. 29, 2007 (IN) 2812/CHE/2007

(51) Int. Cl. A61K 31/437; A61K 9/48; A61P 35/00; A61B 19/00

(52) U.S. Cl. 424/451; 514/283; 206/438

(57) ABSTRACT

There is provided a powder composition for use in a pharmaceutical product, the composition including a) 5(S)-(2′-hydroxyethoxy)-20(S)-CPT; at least one cyclodextrin; wherein 5(S)-(2′-hydroxyethoxy)-20(S)-CPT includes less than 5% of 5(R)-(2′-hydroxyethoxy)-20(S)-CPT. Preferably, in the powder composition, 5(S)-(2′-hydroxyethoxy)-20(S)-CPT is substantially free from said 5(R)-(2′-hydroxyethoxy)-20(S)-CPT.

### INTRODUCTION

The present patent application relates to pharmaceutical compositions of [5(S)-(2′-hydroxyethoxy)-20(S)-camptothecin (referred to as S-isomer of DRF 1042 herein after).

Camptothecin (CPT) is an alkaloid with strong anti-tumour activity isolated from camptotheca acuminate. CPTs are inhibitors of topoisomerase I. CPT and its analogs elicit differential responses in the cell cycle of non-tumorigenic and tumorigenic human cells in-vitro. The only camptothecin analogs to be commercialized to date include topotecan hydrochloride (marketed by GlaxoSmithKline under the brand name HYCAMTIN in vials as a sterile lyophilized powder to be reconstituted before administration to a strength of 4 mg base/ml and also as oral capsules equivalent to 0.25 mg and 1 mg base) and irinotecan (marketed by Pharmacia and Upjohn under the brand name CAMPTOSAR® injection at a strength of 20 mg/ml irinotecan hydrochloride, 2 ml and 5 ml vials).

CPTs containing an α-hydroxy-δ-lactone ring functionality, believed to be essential for the anticancer activity of CPTs, were found to undergo hydrolysis under physiological conditions to form a ring-opened form of the CPT (also known as the carboxylate form) which is less effective therapeutically, has a significantly shorter plasma half-life and is more toxic than the closed lactone form [Hertzberg et al., J. Med. Chem., 32, 715 (1989); J. M. Covey, C. Jaxel et al., Cancer Research, 49, 5016 (1989); Giovanella et al., Cancer Research, 51, 3052 (1991)].

Formation of inclusion complexes for various CPT analogs other than DRF 1042 or its isomers had been described, for example, in U.S. Patent Application Publication No. 2006/0025380, U.S. Patent Application Publication No. 2005/0209190, U.S. Pat. No. 6,653,319, and PCT Application Publication No. WO 2007/018943.

U.S. Pat. No. 6,653,319 describes the formation of inclusion complexes of DB-67 (silatecan) by preparation of a ring-opened species of the compound by complete dissolution in an alkaline medium.

U.S. Patent application Publication No. 2006/0025380 describes non-parenteral formulations using hydrophobic cyclodextrins.

U.S. Patent application Publication No. 2005/0209190 covers cyclodextrin complexes of camptothecin analogs such as 9-nitro camptothecin.

There is a need for stable compositions of S-isomer of DRF-1042 with improved solubility/dissolution characteristics that help in the effective delivery of S-isomer of DRF-1042, pharmaceutical formulations and processes to prepare such compositions and formulations.

### SUMMARY

In one aspect, there is provided a powder composition for use in a pharmaceutical product, said composition including a) 5(S)-(2′-hydroxyethoxy)-20(S)-CPT; and b) at least one cyclodextrin, wherein 5(S)-(2′-hydroxyethoxy)-20(S)-CPT includes less than 5% of 5(R)-(2′-hydroxyethoxy)-20(S)-CPT. Preferably, (S)-(2′-hydroxyethoxy)-20(S)-CPT is substantially free from 5(R)-(2′-hydroxyethoxy)-20(S)-CPT. Various embodiments and variants are provided.

In another aspect, there is provided a pharmaceutical formulation for oral administration that includes a therapeutically effective dose of 5(S)-(2′-hydroxyethoxy)-20(S)-CPT in the form of the powder composition described herein. Various embodiments and variants are provided.

In one aspect, there is provided a pharmaceutical formulation for parenteral administration including i) a therapeutically effective dose of 5(S)-(2′-hydroxyethoxy)-20(S)-CPT in the form of the powder composition of claim 1; and ii) a container suitable for a parenteral pharmaceutical product. Various embodiments and variants are provided.

In another aspect, there is provided a kit including a pharmaceutical formulation for parenteral administration, said kit including: i) a therapeutically effective dose of 5(S)-(2′-hydroxyethoxy)-20(S)-CPT in the form of the powder composition described herein; and ii) a pharmaceutically acceptable diluent for reconstitution.

In another aspect, there is provided a pharmaceutical formulation for parenteral administration including i) a therapeutically effective dose of 5(S)-(2′-hydroxyethoxy)-20(S)-CPT, which is substantially free from 5(R)-(2′-hydroxyethoxy)-20(S)-CPT, and a cyclodextrin in the form of a sterile solution in a vehicle suitable for parenteral administration, said 5(S)-(2′-hydroxyethoxy)-20(S)-CPT and said cyclodextrin being dissolved in said vehicle; and ii) a container suitable for a parenteral pharmaceutical product.

In another aspect, there is provided a method of making a powder composition that includes 5(S)-(2′-hydroxyethoxy)-20(S)-CPT and a cyclodextrin, said method including:

a) providing a solution or dispersion of 5(S)-(2′-hydroxyethoxy)-20(S)-CPT which is substantially free from 5(R)-(2′-hydroxyethoxy)-20(S)-CPT and at least one cyclodextrin in a solvent; b) combining said solution or dispersion with a complexation enhancer; and c) removing said solvent; thereby providing said powder composition.

### BRIEF DESCRIPTION OF FIGURES

FIG. 1 provides the phase solubility curves for S-isomer of DRF-1042 with different concentrations of aqueous HPBCD.

FIG. 2 provides the pH-solubility profile for S-isomer of DRF 1042.

FIG. 3 is the comparative dissolution profile for the compositions of Example 6, Example 9A and Example 12 in fasted state simulated gastric fluid (0.1 N HCl) when tested in USP Type II apparatus, 50 rpm.

FIG. 4 is the X-Ray Powder Diffractogram (XRPD) of S isomer of DRF 1042, physical mixture of S-isomer of DRF 1042 and excipients, placebo and powder composition of Example 7. The terms in the figure represent XRPD of
A: the solubilizing composition
B: the placebo
C: the physical mixture of S-isomer of DRF 1042 and excipients of the solubilizing composition
D: S-isomer of DRF 1042

FIG. 5 is the XRPD of S-isomer of DRF 1042, physical mixture of S-isomer of DRF 1042 and excipients, placebo and solubilizing composition of Example 13. The terms in the figure represent XRPD of
E: S-isomer of DRF 1042
F: the solubilizing composition from Example 13
G: the placebo of the solubilizing composition (Example 13 composition without S-isomer of DRF 1042
H: hydroxypropyl beta cyclodextrin (HPBCD).
I: the physical mixture of S-isomer of DRF 1042 and excipients of the solubilizing composition.

FIG. 6 provides an example of the release profile of S-isomer of DRF 1042 from the composition of Example 17.

FIG. 7: XRPD of lyophilized HPBCD used in Example 20.

FIG. 8: XRPD of the lyophilized placebo of Example 20.

FIG. 9: XRPD of the lyophilized compositions of Example 20.

### DETAILED DESCRIPTION

DRF-1042 is a C-5 substituted analog of 20(S)-CPT intended for the treatment of solid refractory tumors such as ovarian cancer, osteosarcoma, leukemia, lymphoma, non-small cell lung cancer, cancer of the central nervous system, breast, colon, or renal cancer. DRF-1042 in the form of a mixture of diastereomers is disclosed in co-assigned U.S. Pat. No. 6,177,439, which is incorporated herein by reference in its entirety and for the specific purpose of disclosing the mixture of diastereomers and methods for preparation of the mixture of diastereomers.

The inventors of the present patent application have discovered that development of a formulation for the S-isomer of DRF 1042 presents significant challenges.

The dissolution rate and solubility of a pharmaceutical compound play an important role in the absorption of the compound when administered orally. These properties are also important for parenteral administration. S-isomer of DRF 1042 is very poorly soluble in water in a free state. S-isomer of DRF 1042 also exhibits poor solubility in bio-relevant media, such as for example at a gastric pH of 1.2 or intestinal pH of 6.8. The drug is also chemically unstable in aqueous solutions. Solubility of S-isomer of DRF 1042 across the physiological pH range is low and pH-dependent, with higher solubility in the alkaline pH range, associated with a significant chemical instability in alkaline conditions due to the almost complete and irreversible conversion of the S-isomer into the R-isomer and formation of the decarboxylated impurity due to hydrolysis.

Hence, design of pharmaceutical formulations of S-isomer of DRF 1042 is a definitive challenge to a formulation scientist. The inventors addressed this challenge, finding solutions that greatly improve the solubility and dissolution rate of the drug.

The term “substantially free of” is hereby incorporated by reference from co-pending and co-assigned U.S. patent application Ser. No. 11/753,432. Specifically, 5(S)-(2′-hydroxyethoxy)-20(S)-camptothecin is substantially free of 5(R)-(2′-hydroxyethoxy)-20(S)-camptothecin if the amount of 5(R)-(2′-hydroxyethoxy)-20(S)-camptothecin present in a mixture that contains both 5(S)-(2′-hydroxyethoxy)-20(S)-camptothecin and 5(R)-(2′-hydroxyethoxy)-20(S)-camptothecin is less than about 2% by weight of the total weight of the mixture. The amount of 5(R)-(2′-hydroxyethoxy)-20(S)-camptothecin in the mixture may be less than about 1.5% w/w, or it may be less than about 1%, and it may be less than about 0.5%, or even less than 0.1% W/W.

The terms “S-isomer of DRF-1042”, and “DRF-(5S,20S)-1042”, as used in the present patent application, include a free form of the compound, its pharmaceutically acceptable salts or the combinations thereof or any crystalline form or amorphous form or combination thereof of the base or pharmaceutically acceptable salts or combinations thereof. Unless expressly specified to the contrary, all such crystalline modifications of the drug substance or it isomers are included within the scope of this term.

The term “powder compositions” as used herein refers to compositions of S-isomer of DRF 1042, either alone or along with other pharmaceutically acceptable excipients, in the powdered form. The term “powder composition” is used in the broadest possible meaning to encompass powder materials that help achieve the objectives of this invention.

The terms “pharmaceutical formulations or pharmaceutical compositions” are used interchangeably and as used herein are intended to include formulations for drug delivery comprising the powder compositions of the invention. Such pharmaceutical formulations could include for example oral dosage forms such as tablets, granules, powders for reconstitution, capsules, caplets, soft gelatin capsules, gelcaps, solutions, suspensions, syrups and the like or dosage forms for parenteral administration such as solutions, dispersions, suspensions or emulsions for injection, lyophilized products or sterile powders for reconstitution and the like, without limitation.

As used herein, “a cyclodextrin” refers to the natural cyclodextrins, α-cyclodextrin, β-cyclodextrin, and Γ-cyclodextrin, and their respective synthetic and semisynthetic derivatives.

The term “CPT-related impurities” denotes compounds having a campthotecin structural skeleton or compounds resulting from the decomposition of compounds having a campthotecin structural skeleton.

The present patent application provides powder compositions that include a) 5(S)-(2′-hydroxyethoxy)-20(S)-CPT, and b) at least one cyclodextrin. As will be described below, the powder composition may be used in various pharmaceutical formulations for oral or parenteral administration.

The S-isomer of DRF-1042, which is described chemically as 5(S)-(2′-hydroxyethoxy)-20(S)-CPT or 4-(S)-Ethyl-4-hydroxy-12-(S)-(2-hydroxyethoxy)-1,12-dihydro-4H-2oxa-6,12a-diazadibenzo-3,13-dione has the structural formula 1.

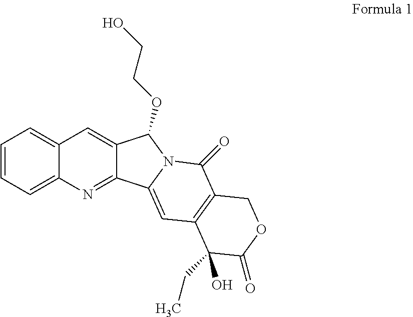

The S-isomer of DRF 1042 is described in detail in co-pending and co-assigned U.S. patent application Ser. Nos. 11/753,432 and 11/753,392, which are incorporated herein by reference in their entirety and for the purposes stated herein below specifically.

As described in U.S. patent application Ser. Nos. 11/753,432, it has been unexpectedly discovered that 5(S)-(2′-hydroxyethoxy)-20(S)-CPT is a significantly better inhibitor of topoisomerase I than either the mixture of diastereomers of DRF 1042 or 5(R)-(2′-hydroxyethoxy)-20(S)-CPT. 5(S)-(2′-hydroxyethoxy)-20(S)-CPT also possesses significant efficacy advantages in various models. All information in U.S. patent application Ser. Nos. 11/753,432 and 11/753,392 that relates to this significant advantage is hereby incorporated by reference for the purpose stated.

Thus, S-isomer of DRF 1042 included in the compositions and formulations described herein, and particularly in powder composition, contains less than 5% of 5(R)-(2′-hydroxyethoxy)-20(S)-CPT. Preferably, S-isomer of DRF 1042 is substantially free of (R)-(2′-hydroxyethoxy)-20(S)-CPT.

The S-isomer of DRF 1042 is the biologically active ingredient of the powder composition, as well as any pharmaceutical product in which it is present or from which it is prepared. Therefore, the amount of S-isomer of DRF 1042 in the powder composition is commensurate with the desired therapeutically effective dose. The dose information is provided further below with respect to description of pharmaceutical formulations.

The powder composition also includes a cyclodextrin. Any cyclodextrin which enhances the aqueous solubility and/or provides for effective delivery of a S-isomer of DRF 1042 compound may be used. Suitable cyclodextrins may include the naturally occurring cyclodextrins and their synthetic or semisynthetic derivatives or their mixtures. The natural cyclodextrins include α-cyclodextrin, β-cyclodextrin and Γ-cyclodextrin. Derivatives are typically prepared by modifying the hydroxyl groups located on the exterior or hydrophilic side of the cyclodextrin. The modifications can be made to increase the aqueous solubility and the stability of the complex and can modify the physical characteristics of the complex including the formation and dissociation of the complex. The types and degree of modification, as well as their preparation, are well known in the art. See, for example, Szejtli, J., Cyclodextrins and Their Inclusion Complexes, Akademiai Kiado: Budapest, 1982; U.S. Pat. Nos. 5,024,998; 5,874,418 and 5,660,845, and references contained therein, all of which are incorporated herein by reference in their entirety and for the purpose stated. Any of the natural cyclodextrins can be derivatized, such as derivatives of β-cyclodextrin. Cyclodextrin derivatives include alkylated cyclodextrins, comprising methyl-, dimethyl-, dimethyl- and ethyl-β-cyclodextrins; hydroxyalkylated cyclodextrins, including hydroxyethyl-, hydroxypropyl-, and dihydroxypropyl-β-cyclodextrin; ethylcarboxymethyl cyclodextrins; sulfate, sulfonate and sulfoalkyl cyclodextrins, such as β-cyclodextrin sulfate, β-cyclodextrin sulfonate, and β-cyclodextrin sulfobutyl ether; as well as polymeric cyclodextrins. Other cyclodextrin derivatives can be made by substitution of the hydroxy groups with saccharides, such as glucosyl- and maltosyl-β-cyclodextrin. Other cyclodextrins include the naturally occurring cyclodextrins, methyl-β-cyclodextrin, dimethyl-β-cyclodextrin, trimethyl-β-cyclodextrin, 2-hydroxymethyl-β-cyclodextrin, hydroxyethyl-β-cyclodextrin, 2-hydroxypropyl-β-cyclodextrin, 3-hydroxypropyl-β-cyclodextrin, β-cyclodextrin sulfate, β-cyclodextrin sulfonate, or β-cyclodextrin sulfobutyl ether. Any of the above cyclodextrins or their derivatives or polymers prepared from them could be used for preparation of the powder compositions of the invention, either alone or in the form of mixtures of one or more cyclodextrins.

Commercially available cyclodextrins may be used such as available from any of the commercial suppliers such as for example M/s CARGILL, M/s ROQUETTE, Aldrich Chemical Company, Milwaukee Wis. and Wacker Chemicals, New Canaan, Conn. or may be synthesized in-house by any of the processes known in the art for the synthesis of cyclodextrins and their derivatives. The synthetic cyclodextrins such as HPBCD and sulfobutylether cyclodextrins among others are preferred due to their proven use in pharmaceutical formulations for administration to human beings, their acceptability to the regulatory authorities, their high aqueous solubility and low toxicity. Hydrophilic cyclodextrins are preferred. Particularly preferred is hydroxypropyl β-cyclodextrin (HPβCD or HPBCD).

The amount of cyclodextrin is selected based on the amount of S-isomer of DRF-1042. The weight ratio of S-isomer of DRF-1042 to cyclodextrin may vary from about 1:1 to about 1:15, preferably, from about 1:5 to about 1:10.

While the invention is not limited by any specific theory, it is believed the component of the composition may form an inclusion complex with one another. The true inclusion complexes of S-isomer of DRF 1042 with HPβCD provide an increase in the aqueous solubility as well as solubility in bio-relevant media of DRF-1042 of more than 50-fold when compared with the solubility of S-isomer of DRF 1042 alone in an uncomplexed state. Such an enhancement in the aqueous solubility and in bio-relevant media is believed to result in significantly improved pharmacokinetic properties, with faster absorption providing higher levels of this potent anticancer agent when given orally, as well as a more complete absorption defined by the bioavailability when compared with the intravenous administration.

Cyclodextrins with lipophilic inner cavities and hydrophilic outer surfaces are capable of interacting with a large variety of guest molecules to form non-covalent inclusion complexes. The stability of the complex formed depends on how well the guest molecule fits into the cyclodextrin cavity. Without being bound by any specific theory, it is believed that the processing of the lipophilic active along with the cyclodextrin provides a composition wherein the active is in intimate contact with the cyclodextrin though not in the form of an inclusion complex. Thus, upon coming in contact with bio-relevant media, the active is forced into solution along with the cyclodextrin.

Formation of the inclusion complex in solution can be evaluated by suitable analytical techniques, for example, UV spectroscopy, circular dichroism, fluorescence spectroscopy, nuclear magnetic resonance, and potentiometry. Solid inclusion complexes may also be studied by measuring solubility in water or bio-relevant media, powder X-ray diffractometry, differential scanning calorimetry or thermogravimetry and the like.

Free powder of DRF-(5S,20S)-1042 may be characterized by its XRPD pattern with significant peaks at about 7.2±0.1, 9.4±0.1, 11.02±0.1, 12.00±0.1, 14.54±0.1, 15.2±0.1, 18.92±0.1, 21.86±0.1, 22.74±0.1 and 26.42±0.1 degrees 2θ. The X-ray diffraction pattern for an exemplary crystalline form of DRF-(5S,20S)-1042 had been set forth in U.S. patent application Ser. No. 11/753,392, which is hereby incorporated by reference for the purpose stated. The presence of the characteristic peaks of the free form of DRF-(5S,20S)-1042 in the XRD of a physical mixture (drug and HPBCD blended in a dry state) and their absence from the powder compositions prepared as described herein indicate the existence of complexation between DRF-(5S,20S)-1042 and cyclodextrin. The inclusion complex of DRF-(5S,20S)-1042 with HPβCD is described by a faint halo when characterized by powder X-ray diffraction (XRPD) indicating the amorphous nature of the inclusion complex and absence of any crystalline drug substance as demonstrated in FIG. 4 and FIG. 5.

It is desirable for S-isomer of DRF 1042 to be present in the form of an inclusion complex with little or no uncomplexed drug present in the solubilizing compositions of the invention. Preferably, S-isomer of DRF 1042 is at least about 70%, or about 75%, or about 80% or about 85% or about 90%, or about 95% or about 100% complexed. The percentage of uncomplexed drug may be determined by quantitative XRPD analysis of the powder composition or by measuring the differences in solubility of the powder compositions in a bio-relevant medium. However, the complexation of S-isomer of DRF 1042 with cyclodextrin may be complete or partial, and both variants are contemplated.

The uncomplexed drug when present in the powder compositions could either be in a crystalline form or in an amorphous form. The crystalline form could be the same as the one which was used in the preparation of the powder compositions or a different crystalline form or mixture of forms could be present.

The powder compositions possess significantly enhanced aqueous solubility and dissolution rates of S-isomer of DRF 1042 in comparison to solubility of S-isomer of DRF 1042 in the free state. The solubility of DRF-(5S,20S)-1042 has been enhanced by about 2000 folds by converting DRF-(5S,20S)-1042 into the powder composition. In particular, preferably, the powder compositions have solubility greater than 5 mg per ml of pure water, more preferably, more than 25 mg per ml. The enhanced solubility is believed to result in a higher in vitro/in vivo dissolution rate in bio-relevant media leading to significantly modified pharmacokinetic parameters.

The powder composition possesses a controlled amount of residual moisture. It is believed that the residual moisture level impacts storage stability of the composition at a desired temperature and duration. The amount of residual moisture present in the powder composition produced as described herein below may range from about 2% to about 8%. Desirably, the amount of residual moisture in the composition is less than about 6% w/w or less than about 4% w/w. Since S-isomer of DRF 1042 is sensitive to the presence of moisture, the powder compositions provide stable S-isomer of DRF 1042 compositions for human use.

Stability may be further enhanced through the use of appropriate packaging conditions to exclude moisture from coming in contact with the dried powder compositions prepared as described above. The preparation of the powder compositions is described below. Either that should be moved up or this statement should be changed to reflect this. Powder compositions may be stored in polyethylene bags, aluminum pouches, polyethylene lined aluminum pouches, containers such as corrugated boxes, fiber, LOPE (low density polyethylene) or HDPE (high density polyethylene) containers lined with any one or more above mentioned bags, either tied or sealed with or without inert gas purging into the packing. Depending on the size of the pack and the quantity of the material in the pack other accessories such as molecular sieves, silica bags, free radical scavengers that aid in stabilization of the products are used.

The powder compositions preferably contain controlled amounts of CPT-related impurities. S-isomer of DRF 1042 is sensitive to moisture, temperature conditions as well as alkaline pH conditions, resulting in the formation of certain impurities. The regulatory authorities require that for a pharmaceutical composition to be administered to patients, the composition should be of sufficient purity with impurity levels below certain prescribed levels upon storage under stipulated conditions for the shelf-life. The impurities that are of particular mention include:

a) R-isomer of DRF 1042:

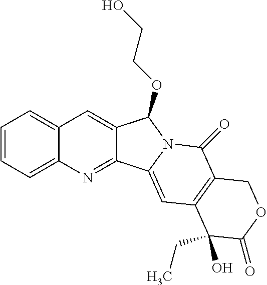

b) a decarboxylated impurity of the chemical formula:

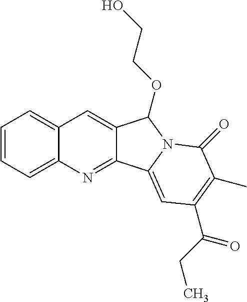

c) a dimer impurity of chemical formula:

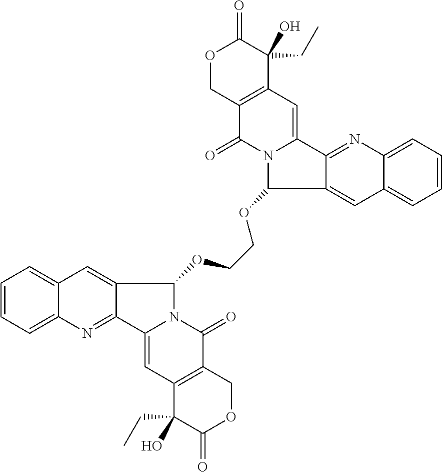

d) dehydro impurity of the chemical formula:

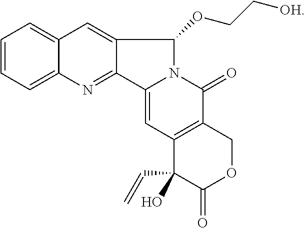

Preferably, the powder compositions contain less than 4% of total CPT-related impurities, more preferably, less than 1%. It is also preferred that the powder compositions contain less than 4% of each individual CPT-related impurity, including impurities a), b), c), and/or d), more preferably, less than 1%. This can be accomplished by providing S-isomer of DRF 1042 substantially free of the impurity and subsequently converting this pure material into the powder composition under controlled conditions of temperature and pH to minimize the formation of impurities, including the impurities a), b), c), and/or d).

The impurity contents described herein relate to individual or the total of impurities, as determined by high performance liquid chromatography (“HPLC”), and any residual solvent impurities.

Also provided are powder compositions with defined physicochemical characteristics, such as particle size distribution, span, bulk density, Hausner ratio, aspect ratio, Carr index.

The particle size of a material is generally described in terms of D₁₀, D₅₀, D₉₀, D_{(4,3)} used routinely to describe the particle size or size distribution. It is expressed as volume or weight or surface percentage. Dₓ as used herein is defined as the size of particles where x volume or weight percent of the particles have sizes less than the value given. D_{(4,3)} for example is the volume mean diameter of the S-isomer of DRF 1042 or other powder compositions. D₉₀ for example means that 90% of the particles are below a particle size. Particle size or particle size distribution of the powder compositions of S-isomer of DRF 1042 are determined by the techniques that are known to the person skilled in the art including but not limited to sieve analysis, particle size analysis by laser principle such as Malvern particle size analyzer and the like. Powder compositions of S-isomer of DRF 1042 are preferably fine, uniform and agglomerate free.

In an embodiment, the powder composition has a particle size distribution wherein D₉₀ is less than about 150μ or less than about 100μ or less than about 75μ and D₅₀ is less than about 75μ or less than about 50μ.

Another indication of the physicochemical characteristics of the powder composition is the density properties such as bulk and tapped density. Bulk density is described as untapped or tapped. Untapped bulk density of a substance is the undisturbed packing density of that substance and tapped bulk density relates to the packing density after tapping a bed of substance until no change in the packing density is seen. Bulk density and tapped density can be determined using compendial bulk density apparatus, the method being given in United States Pharmacopeia 29, United States Pharmacopeial Convention, Inc., Rockville, Md., 2005, at pages 2638-2639. A higher bulk density indicates a dense material allowing a higher dose to be filled into a given size capsule for example. The powder compositions can have bulk densities from about 0.8 g/ml to about 0.2 g/ml, or from about 0.6 g/ml to about 0.2 g/ml.

The Hausner ratio is a measure of inter-particle friction and the potential powder arch or bridge strength and stability (Hausner, H. H. Friction conditions in a mass of metal powders. International Journal of Powder Metallurgy 1967, 3 (4), pages 7-13). It has been widely used to estimate the flow properties of powders, blends, granules and other such particles or aggregates and is expressed as the ratio of tapped bulk density to the untapped bulk density of the substance. Hausner ratio used herein is defined as ratio of tapped to untapped bulk densities. A Hausner ratio of <1.2 indicates good flow while ratio >1.5 indicate poor flow. The powder compositions can have a Hausner ratio less than 1.5 or less than 1.2.

Carr index as used herein is defined as the percent compressibility which is a percentage ratio of the difference between tapped bulk density and initial bulk density to tapped bulk density. Carr index values between 5-15% represent materials with excellent flowability, values between 18-21% represent fair-flowability and values above 40% represent very poor flowability. The powder compositions of the invention can have Carr index values less than 40% or less than 21% or less than 15%.

Crystalline content means the ratio of crystalline substance to the total of amorphous S-isomer of DRF 1042. Crystalline content is determined by the techniques known to the persons skilled in the art that includes X-ray powder diffraction, solid state NMR, Fourier Transform Infra-red spectrometry and the like. Preferably, the powder compositions of S-isomer of DRF 1042 are amorphous, wherein the crystalline content is within a range showing no influence on in-vitro release profile.

The powder composition may include complexation enhancers to improve complexation of S-isomer of DRF 1042 with the cyclodextrin. Preferably, the ratio of S-isomer of DRF 1042 to complexation enhancer/s is in the range of about 1:1 to about 1:20 or from about 1:1 to about 1:15 or from about 1:1 to about 1:10 by weight.

Examples of complexation enhancers are surfactants, alkalizing agents, and solubilizing agents. Complexation enhancers in the form of surfactants, alkalizing agents or solubilizers either may be used alone or a combination of two or more may be used for maximum effect.

Surfactants improve the wetting property of the active ingredient. Various useful surfactants include but are not limited to sodium lauryl sulfate, polysorbate 80, poloxamer 188, poloxamer 407, sodium carboxy methylcellulose hydrogenated oil, polyoxyethylene glycol, and polyoxypropylene glycol, polyoxyethylene sorbitan fatty acid esters, polyglycolized glycerides available commercially such as GELUCIRE 40/14, GELUCIRE 42/12, GELUCIRE 50/13, Vitamin E TGPS and so on.

Emulsifying agents can also include any of a wide variety of cationic, anionic, zwitterionic, and amphoteric surfactants such as are known in the art. Non-limiting examples of anionic emulsifying agents include the alkoyl isethionates, alkyl and alkyl ether sulfates and salts thereof, alkyl and alkyl ether phosphates and salts thereof, alkyl methyl taurates, and soaps such as for example alkali metal salts including sodium or potassium salts of long chain fatty acids.

Examples of amphoteric and zwitterionic emulsifying agents are those which are broadly described as derivatives of aliphatic secondary and tertiary amines in which the aliphatic radical can be straight or branched chain and wherein one of the aliphatic substituents contains from about 8 to about 22 carbon atoms and one contains an anionic water solubilizing group, e.g., carboxy, sulfonate, sulfate, phosphate, or phosphonate. Examples are alkyl imino acetates and iminodi alkanoates and aminoalkanoates, imidazolinium and ammonium derivatives. Other suitable amphoteric and zwitterionic emulsifying agents are those selected from the group consisting of betaines, sultaines, hydroxysultaines, alkyl sarcosinates and alkanoyl sarcosinates.

These silicone-emulsifying agents are typically organically modified organopolysiloxanes, also known to those skilled in the art as silicone surfactants. Useful silicone emulsifying agents include dimethicone copolyols. These materials are polydimethyl siloxanes, which have been modified to include polyether side chains such as polyethylene oxide chains, polypropylene oxide chains, mixtures of these chains, and polyether chains containing moieties derived from both ethylene oxide and propylene oxide.

Examples of suitable emulsifying agents include, disodium cocoampho di acetate, oxyethylenated glyceryl cocoate (7 EO), PEG-20 hexadecenyl succinate, PEG-15 stearyl ether; the ricinoleic monoethanolamide monosulfosuccinate salts, oxyethylenated hydrogenated ricinoleic triglyceride containing 60 ethylene oxide units such as the product sold by BASF under the trademarks CREMOPHOR RH60 or CREMOPHOR RH 40 (polyoxyl 40 hydrogenated castor oil), polymers such as Poloxamers, which are block copolymers of ethylene oxide and propylene oxide, and the non-solid fatty substances at room temperature (that is to say at a temperature ranging from about 20 to 35° C.) such as sesame oil, almond oil, apricot stone oil, sunflower oil, octoxyglyceryl palmitate (or 2-ethylhexyl glyceryl ether palmitate), octoxyglyceryl behenate (or 2-ethylhexyl glyceryl ether behenate), dioctyl adipate, tartrate of branched dialcohols.

Non-ionic emulsifying agents include those that can be broadly defined as condensation products of long chain alcohols, e.g. C8-30 alcohols, with sugar or starch polymers, i.e., glycosides. Various sugars include but are not limited to glucose, fructose, mannose, and galactose; and various long chain alcohols include but are not limited to decyl alcohol, cetyl alcohol, stearyl alcohol, lauryl alcohol, myristyl alcohol, oleyl alcohol, and the like. Commercially available examples of this type of emulsifying agents include decyl polyglucoside (available as APG 325 CS from Henkel) and lauryl polyglucoside (available as APG 600 CS and 625 CS from Henkel).

Other useful non-ionic emulsifying agents include the condensation products of alkylene oxides with fatty acids (i.e., alkylene oxide esters of fatty acids). Other non ionic surfactants are the condensation products of alkylene oxides with 2 moles of fatty acids (i.e., alkylene oxide diesters of fatty acids). Other non-ionic emulsifying agents are the condensation products of alkylene oxides with fatty alcohols (i.e., alkylene oxide ethers of fatty alcohols). Still other non-ionic emulsifying agents are the condensation products of alkylene oxides with both fatty acids and fatty alcohols [i.e., wherein the polyalkylene oxide portion is esterified on one end with a fatty acid and etherified (i.e. connected via an ether linkage) on the other end with a fatty alcohol]. Non-limiting examples of these alkylene oxide derived non-ionic emulsifying agents include ceteth-6, ceteth-10, ceteth-12, ceteareth-6, ceteareth-10, ceteareth-12, steareth-6, steareth-10, steareth-12, PEG-6 stearate, PEG-10 stearate, PEG-100 stearate, PEG-12 stearate, PEG-20 glyceryl stearate, PEG-80 glyceryl tallowate, PEG-10 glyceryl stearate, PEG-30 glyceryl cocoate, PEG-80 glyceryl cocoate, PEG-200 glyceryl tallowate, PEG-8 dilaurate, PEG-10. Other non-ionic emulsifying agents include sugar esters and polyesters, alkoxylated sugar esters and polyesters, CI-C30 fatty acid esters of CI-C30 fatty alcohols, alkoxylated ethers of CI-C30 fatty alcohols, polyglyceryl esters of CI-C30 fatty acids, CI-C30 esters of polyols, CI-C30 ethers of polyols, alkyl phosphates, polyoxyalkylene fatty ether phosphates, fatty acid amides, acyl lactylates, and mixtures thereof. Non-limiting examples of these emulsifying agents include: polyethylene glycol 20 sorbitan monolaurate (Polysorbate 20), polyethylene glycol 5 soya sterol, Steareth-20, Ceteareth-20, PPG-2 methyl glucose ether distearate, Ceteth-10, Polysorbate 80, cetyl phosphate, potassium cetyl phosphate, diethanolamine cetyl phosphate, Polysorbate 60, glyceryl stearate, poly oxyethylene 20 sorbitan trioleate (Polysorbate 85), sorbitan monolaurate, poly oxyethylene 4 lauryl ether sodium stearate, polyglyceryl-4 isostearate, hexyl laurate, PPG-2 methyl glucose ether distearate, PEG-100 stearate, and mixtures thereof. Further examples of suitable emulsifiers include mixtures of stearyl octanoate and isopropyl myristate, or mixtures of cetyl octanoate and stearyl octanoate.

Desirable emulsifiers include sodium lauryl sulfate, polysorbate 80, polyglycolized glycerides available commercially grades such as GELUCIRE 40/14, GELUCIRE 42/12, GELUCIRE 50/13, Vitamin E TPGS and the like.

Complexation enhancers may include alkalizing agents, such as, for example, organic amines, such as meglumine, tromethamine, triethanolamine, diethanolamine among others, inorganic alkalies, such as for example sodium hydroxide, sodium carbonate, sodium bicarbonate and the like; amino acids such as for example natural amino acids, including all isomeric forms individually and in racemic and non-racemic mixtures, and analogs of amino acids, including all isomeric forms individually and in racemic and non-racemic mixtures, peptides and polymers of amino acids, their salts with other reactants and further including mixtures of each of the above. Some examples of amino acids include alanine, isoleucine, leucine, methionine, phenylalanine, proline, tryptophan, valine, asparagine, cysteine, glutamine, glycine, serine, threonine, tyrosine, aspartic acid, glutamic acid, arginine, histidine, lysine and the like. The use of mixtures of two or more of the above mentioned alkalizing agents either from the same class or from different classes of alkalizing agents is also within the scope of the invention.

Any alkalizing compound is acceptable as long as they provide a pH value to the solvent medium in the range of interest and is not chemically detrimental to the DRF-1042 or to the complex formed. Alkalizing compounds which provide the desired pH yet are not strong enough to solubilize the active in the alkaline solution thereby formed are particularly important in the preparation of the inclusion complexes of the invention as they allow for the preparation of inclusion complexes of exceptionally high purity.

The powder composition may also include other pharmaceutically acceptable excipients, for example wetting agents, pH modulators, diluents or bulking agents, and the like. The excipients included may be capable of playing more than one role in the preparation of the solubilizing compositions.

Various methods are known in the art to prepare drug:cyclodextrin complexes, including the solution method, co-precipitation method, the slurry method, the kneading method, the grinding method. See T. Loftsson, Pharmaceutical Technology, 1999, 12, 41-50.

In the solution method, the drug, either as a solid or in a solution, is added to a solution containing an excess amount of cyclodextrin. It is also possible to add an excess of the drug to an aqueous cyclodextrin solution. The mixture is agitated, and may optionally be heated, until equilibrium is reached, which may take several hours or several days. The equilibrated solution is then filtered or centrifuged to give a clear solution of the drug-cyclodextrin complex. The clear solution can be directly administered to a subject, or a solid complex can be obtained by removal of the water by evaporation (such as spray-drying), sublimation (such as lyophilization) or other drying means well known in the art.

A solid complex may also be obtained by the precipitation method. Often, the cyclodextrin complexes precipitate upon cooling of the solution. Otherwise, a solvent in which the complex has minimal solubility, typically an organic solvent, is used to precipitate the solid complex. The precipitate containing the complex can then be filtered or centrifuged to obtain a solid drug-cyclodextrin complex. A generally less effective method of preparing a solid complex mixture is to grind a dry mixture of the drug and cyclodextrin in a sealed container, which is then gently heated to a temperature between 60 to 140° C.

If the drug is poorly water-soluble, the slurry or kneading methods can be employed. The drug and cyclodextrin can be suspended in water to form slurry, which is similarly stirred and/or heated to equilibration. The complex can be collected by filtration or by evaporation of the water. The kneading method is similar to the slurry method, whereby the drug and cyclodextrin are mixed with a minimal amount of water to form a paste. The complex can be isolated by methods similar to those discussed above.

The above methods generally utilize an excess amount of cyclodextrin to maximize equilibration of a cyclodextrin:drug complex. The amount of cyclodextrin in the desired formulation is directly related to the amount of the desired drug concentration and the molar ratio of cyclodextrin:drug in the complex.

Similarly, XRPD for active, physical mixture and the complex (partial and complete) are taken and pure active shows crystalline peaks and the number and intensity of peaks disappear as the observation moves towards more complex or complete complex formed samples.

Any method may be used for the preparation of the inclusion complexes described herein including but not limited to the methods described above. According to one embodiment, processes for the preparation of the inclusion complexes of the invention are provided comprising combining a cyclodextrin and DRF-1042 in the desired ratio under suitable conditions, optionally along with other pharmaceutically acceptable excipients that aid or enhance the complexation or act as bulking agents.

In a specific embodiment the invention describes processes to prepare the powder compositions comprising:
a) providing a solution or dispersion comprising DRF-1042 and a cyclodextrin in a suitable solvent medium;
b) adjusting the pH of the solution of step (a) as desired using a pH modulator; and
c) recovering the powder composition from the solution.

In one aspect of this embodiment, the process to prepare powder compositions in the form of inclusion complexes of DRF-1042 comprises the steps of:
a) providing a dispersion of DRF-1042 in a suitable solvent medium;
b) optionally adding a pharmaceutically acceptable bulking agent;
c) adding complexation enhancers to the dispersion of step (a) or step (b) and optionally adjusting the pH as desired;
d) dissolving a cyclodextrin in the dispersion of step (c);
e) mixing the dispersion of step (d) to form a clear solution;
f) adjusting the pH of the clear solution of step (e) as desired using a pH modulator;
g) optionally filtering the solution; and
h) optionally evaporation of the solvent to obtain a dry product.

Step (a) comprises providing a dispersion of DRF-1042 in a suitable solvent medium. DRF-1042 or its individual isomer may be in any crystalline form in which they exist or as an amorphous material, without limitation. Also, the use of mixtures of crystalline forms or isomeric forms is within the scope of the invention.

It is desirable, though not absolutely essential, that the active be of as small a particle size as possible before being added to the solvent medium. A smaller particle size enhances the speed of dissolution of a solid in a given solvent medium. Also, a smaller particle size enhances the suspendability in the medium when the method of preparation of the inclusion complex involves the preparation of a dispersion of the active in the solvent medium. In addition, a smaller particle size also reduces the time required for complexation. The particles of the active may thus be of a mean particle size of less than about 500 μm or about 350 μm or about 200 μm or about 150 μm or about 100 μm or about 50 μm or about 25 μm or lower than this size. The fine particles prepared according to the procedures described herein also form another embodiment of these inventive powder compositions of S-isomer of DRF 1042.

The particle size may be reduced to the desired level by any method of size reduction known in the art such as for example pulverization, air jet milling (using compressed air), ball milling, and the like without limitation. Alternatively, larger particles can be added to the medium and the slurry can be subjected to homogenization using for example a high speed homogenizer, a high pressure homogenizer, colloid milling, emulsiflex, microfluidizer, bead mill and the like without limitation. Other methods of size reduction are well within the scope of this invention.

The solvent medium used in the preparation of the inclusion complexes include but are not limited to water, methanol, ethanol, acidified ethanol, acetone, diacetone, polyols, polyethers, oils, esters, alkyl ketones, acetonitrile, methylene chloride, isopropyl alcohol, butyl alcohol, methyl acetate, ethyl acetate, isopropyl acetate, castor oil, ethylene glycol monoethyl ether, diethylene glycol monobutyl ether, diethylene glycol monoethyl ether, dimethyl sulphoxide, dimethyl formamide, tetrahydrofuran and mixtures thereof.

In one embodiment, water or mixtures of water with different water-miscible organic solvents are used for the preparation of the inventive inclusion complexes. Any solvent medium is acceptable for the preparation of the inclusion complexes of the invention as long as the active is soluble or dispersible in the medium, the cyclodextrin is soluble in the medium and the medium is not detrimental to the active or the complex formed, chemically.

The ratio of the solvent medium to the active will be decided by the final concentration of the S-isomer of DRF 1042, which is to be achieved in solution in the form of a complex and the cyclodextrin that is to be used, which can be deduced by routine experimentation by a person skilled in the art of preparation of inclusion complexes. As a routine practice, solutions of the cyclodextrin in the solvent medium, in water for example, are prepared in different concentrations. To these solutions are added different amounts of S-isomer of DRF 1042 and the suspensions are allowed to equilibrate aided by shaking. The suspensions are subsequently filtered and analyzed for content of S-isomer of DRF 1042.

The temperature of the solvent medium is preferably kept at about room temperature though higher or lower temperatures may be used as required. Any temperature is acceptable as long as it is not detrimental to the chemical stability of the active, the cyclodextrin and to the stability of the inclusion complex formed.

Step (b) involves the addition of a pharmaceutically acceptable bulking agent. Examples of bulking agents include but are not limited to sodium chloride, mannitol and other pharmaceutically acceptable sugars. The ratio of S-isomer of DRF 1042 to bulking agent(s) may range from about 1:1 to about 1:25, or from about 1:1 to about 1:15 or from about 1:1 to about 1:10 by weight, applicable for all aspects and embodiments described in the present patent application. By including a bulking agent in the complex solution, drug loss during process of spray drying can be reduced. Further, the presence of a bulking agent is useful in modifying the physicochemical properties of the powder compositions such as bulk density, which determine the amount of active that can be incorporated into the pharmaceutical delivery vehicle such as for example a capsule. Additionally, the inclusion of a suitable pharmaceutically acceptable bulking agent allows the preparation of a product, which is ready to fill into a capsule or compress into tablets, with appropriate flow properties and compressibility. In the case of a lyophilized product for example, the bulking agent allows the final solution of the inclusion complex to be lyophilized to provide a product cake with aesthetic appeal. Suitable pharmaceutically acceptable bulking agents could include for example mannitol, sodium chloride, sucrose, glucose, lactose, dextrose, dextrins and the like.

Step (c) involves the addition of complexation enhancers to the dispersion of step (b) and adjusting the pH as desired. The complete complexation is believed to occur when the pH of the medium is above 6.

In one aspect of step (c), the pH of the dispersion may be adjusted in the required range. An alkaline pH is generally desirable due to the high aqueous solubility of S-isomer of DRF-1042 in alkaline conditions. The pH can be adjusted in the range of between about 7 to about 14 or about 8 to about 12. Any pH is acceptable as long as it is not detrimental to the chemical stability of S-isomer of DRF-1042. Any of the alkalizing agents mentioned above can be used for adjusting the pH in the desired range or a combination of alkalizing agents can be used.

It is observed that S-isomer of DRF-1042 is unstable in alkaline conditions resulting in rapid and extensive degradation in these media. It is surprisingly observed that DRF-1042 is insoluble in alkaline media where the pH is adjusted by using an amino acid, yet allows the preparation of the inclusion complexes.

Thus, according to this embodiment, the S-isomer of DRF-1042 is in suspension even when the pH is adjusted to between about 8 to 10 using an amino acid. Any amino acid is acceptable as long as it provides an alkaline pH as described above. Arginine, lysine and histidine are particularly desirable for this purpose. It is also surprisingly observed that even though the S-isomer of DRF-1042 are not in solution in the aqueous medium, formation of the inclusion complex is always complete when prepared by the process of the invention. Additionally, surprisingly, the process of the invention where an amino acid is used provides powder compositions of exceptionally high purity and stability. This formation of the inclusion complexes of S-isomer of DRF-1042 even though the active is not in solution before addition of the cyclodextrin thus forms an important embodiment of this invention.

Step (d) involves the dissolution of a cyclodextrin in the dispersion of step (c). Step (e) involves mixing of the dispersion of step (d) to form a clear solution. Any means of mixing dispersions is acceptable as long as it provides a clear solution of S-isomer of DRF-1042 in the aqueous medium. Such mixing means could include for example overhead stirrers, homogenizers, static mixers, sonicators and the like. The duration of mixing will be decided based on parameters such as concentration to be achieved, the temperature of the dispersion, the type of cyclodextrin, the mixing means, the particle size of the S-isomer of DRF-1042 in the dispersion and such other parameters known to a person skilled in the art of preparing inclusion complexes. The temperature of the dispersion may be increased to enhance the rate of formation of the inclusion complex. A temperature in the range of about 20° C. to about 70° C. or about 20° C. to about 40° C. is generally acceptable, though lower or higher temperatures are well within the scope of the invention. Any temperature is acceptable as long as it is not detrimental to the chemical stability of the active or the complex formed.

It is important to ensure that a clear solution is achieved before the mixing is discontinued as this is an indication of completeness of formation of the inclusion complex.

Step (f) involves adjusting the pH of the clear solution of step (e) as desired using a pH modulator. The pH may be adjusted in a range of for example neutral to slightly acidic such as from about 4 to about 8 or about 5 to about 7.5. It is preferable to add an aqueous solution of an acid such as for example hydrochloric acid, sulfuric, phosphoric, nitric acids among other acids, though acids could be added directly to the solution of step (e) as well. The adjustment of the pH to the appropriate range for the active compound to provide an inclusion complex of exceptional purity and stability is an important embodiment of the invention.

Steps (g) and (h) involve filtering the solution of step (f) and further evaporation of the solvent to obtain a dry product.

The clear solution obtained as described above may be filtered to remove extraneous material or undissolved drug substance to prevent these from getting into the final product. Any filter medium may be chosen such as for example different grades of membrane filters, sintered glass filters and the like.

In an embodiment the filtrate may be used as a solution for injection or may be reconstituted or diluted prior to parenteral administration. It is understood that when the solution is to be used for injection the solution will be processed as per the requirements for producing a sterile and endotoxin free product. Such processes are well known in the art of manufacturing pharmaceutical sterile dosage forms.

The filtered solution may optionally be subjected to evaporation of the solvent medium to recover a dry product. Any method of solvent evaporation or drying is acceptable as long as it is not detrimental to the chemical stability of the drug as well as the solubilizing composition. Such methods could include for example tray drying, vacuum drying, spray drying, lyophilization, microwave drying and the like without limitation. Two or more methods could be used sequentially to ensure completeness of removal of the solvent medium or to achieve desirable bulk properties of the dried solubilizing compositions. Thus, according to one particular embodiment, the inclusion complex solutions as prepared above are spray dried and the resulting powder is optionally further subjected to vacuum drying to get the desired moisture content.

Thus according to this embodiment of the invention, the inclusion complex solution as prepared above is further subjected to spray drying to obtain a dry product which constitutes one of the powder compositions described herein. Spray drying is a drying technique of particular interest in the preparation of dry powder compositions of the invention due to its rapid drying cycles, high throughputs, scalability, short exposure times to high temperatures, achievement of desired bulk properties and other reasons. S-isomer of DRF-1042 is sensitive to temperature and moisture. Thus, for the preparation of the dry complex, appropriate control over the drying conditions provides dry powder compositions of exceptionally high purity and stability. Modification of the drying conditions such as feed concentration, rate of spraying during drying, atomization pressure which determines the droplet size, presence or absence of bulking agents and other parameters allow a pharmaceutical scientist to obtain a product with varied moisture contents, bulk densities and other properties.

A dry powder composition prepared as described can further be subjected to vacuum drying to further remove the residual moisture.

In another embodiment, there are provided compositions of S-isomer of DRF-1042 which are in a lyophilized form and which may be used as is or upon reconstitution with aqueous media provides a pharmaceutical formulation in a form of solution for injection that is ready for administration by parenteral route.

The technique known as lyophilization can be employed for injectable pharmaceuticals, which exhibit poor stability in aqueous solutions. Lyophilization process is suitable for injectables because it can be processed in sterile conditions, which is primary requirement for parenteral dosage forms. During the lyophilization process, the complex structure could become damaged leading to leakage of drug. Such damage could be prevented by the use of cryoprotectants. Cryoprotectants as per the present invention include all the bulking agents which may be used in the invention.

Lyophilization or freeze drying is a process in which water is removed from a product after it is frozen and placed under a vacuum, allowing the ice to change directly from solid to vapor without passing through a liquid phase. The process consists of three separate, unique, and interdependent processes; freezing phase, primary drying phase (sublimation), and secondary drying phase (desorption). These processes may be optimized to enhance the product stability as well as decrease the manufacturing costs.

### Freezing Phase:

The primary function of the freezing phase is to ensure that the entire container with the complex solution is completely frozen prior to proceeding to the primary dry phase. Additionally, it is preferable that these containers freeze in a uniform manner. While there are different ways that this can be accomplished, one option is to chill the containers after they are loaded onto the lyophilizer shelves and held for 30-60 minutes prior to initiation of the freezing cycle. It is generally not practical to equilibrate the shelves to a freezing temperature, because of frost accumulation during the filling and loading of the containers.

### Primary Drying Phase:

Once the formulation is brought to the desired frozen state, primary drying via sublimation can proceed. The primary dry phase involves the removal of bulk water at a product temperature below the ice transition temperature under a vacuum (pressures typically between 50-150 mTorr). This phase is the most critical one for stabilizing active. The goal of this testing is to identify the glass transition temperature (Tg′) for the formulation. The Tg′ is the temperature at which there is a reversible change of state between a viscous liquid and a rigid, amorphous glassy state One can measure the Tg′ of candidate formulations using a differential scanning calorimeter (DSC), in particular with modulated DSC. Generally, the collapse temperature is observed to be about 2-5° C. greater than the Tg′. Hence, the shelf temperature is set such that the target product temperature is maintained near or below the Tg′ of the formulation throughout the removal of solvent during the primary dry phase.

As the solvent is progressively removed from the formulated containers, the product temperature will approach and reach the shelf temperature since it is no longer cooled by water sublimation. To optimize the duration of the primary dry phase, the removal of solvent vapor can be tracked using a moisture detector, or by monitoring the decrease in pressure difference between a capacitance manometer and a thermocouple pressure gauge or by a pressure drop measurement. The optimization of the primary dry cycle involves the removal of solvent as quickly as possible without causing cake collapse and subsequent product instability.

### Secondary (Terminal) Dry Phase:

Secondary dry phase is the final segment of the lyophilization cycle where residual moisture is removed from the formulation interstitial matrix by desorption with elevated temperature and/or reduced pressure. The final moisture of a lyophilized formulation, which can be measured by Karl Fisher or other methods, is important to determine because if the cake contains too much residual moisture, the stability of the active can be compromised. Hence, it is imperative that one achieves a moisture level less as possible.

To accomplish a low residual moisture, the shelf temperature is typically elevated to accelerate desorption of water molecules. The duration of the secondary dry phase is usually short. When microstructure collapse occurs, the residual moisture is generally significantly greater than desired. One alternative is to purge the sample chamber of the lyophilizer with alternating cycles of nitrogen to facilitate displacement of bound water. However, the best solution is to properly formulate the drug product and run an optimal lyophilization cycle.

The advantages of lyophilization include: Ease of processing a liquid, which simplifies aseptic handling; Enhanced stability of a dry powder; Removal of water without excessive heating of the product; Enhanced product stability in a dry state; Rapid and easy dissolution of reconstituted product. And also the product is dried without elevated temperatures thereby eliminating adverse thermal effects; and the stored in the dry state in which there are relatively few stability problems.

Additionally freeze dried products are often more soluble and/or more rapidly powder, dispersions are stabilized, and products subject to degradation by oxidation or hydrolysis are protected.

The lyophilization process generally includes the following steps:
1) Providing the complex solution prepared as discussed above.
2) Sterilizing the bulk solution by aseptic filtration.
3) Filling into individual sterile containers and partially stoppering the containers under aseptic conditions.
4) Transporting the partially stoppered containers to the lyophilizes and loading into the chamber under aseptic conditions.
5) Applying the lyophilization cycle comprising freezing phase, primary drying and secondary drying. Freezing the solution by placing the partially stoppered containers on cooled shelves in a freeze-drying chamber or pre-freezing in another chamber.
6) Applying a vacuum to the chamber and heating the shelves in order to evaporate the water from the frozen state.
7) Stoppering of the vials usually by hydraulic or screw rod stoppering mechanisms installed in the lyophilizers.

Pharmaceuticals to be freeze dried are usually in aqueous solution ranging from 0.01 to 40% in concentration of total solids. Usually the improvement in stability of the lyophilizate, compared to the solution, is due to the absence of water in the pharmaceutical composition.

The active constituent of many pharmaceutical products, though is present in such a small quantity that if freeze dried alone, it may not give a composition of suitable bulk and in some cases its presence would be hard to detect visually. Therefore excipients are often added to increase the amount of solids present. In most applications it is desirable for the dried product cake to occupy essentially the same volume as that of the original solution. To achieve this, the total solids content of the original solution is usually about 10 to 25%.

Among substances found useful for this purpose, often in combination are sodium or potassium phosphates, citric acid, tartaric acid, gelatin, lactose and toehre carbohydrates such as dextrose, mannitol and dextran and on occasion, preservatives. Various excipients contribute appearance characteristics to the cake, such as whether dull and spongy or sparkling and crystalline, firm or friable, expanded or shrunken, and uniform or striated. Therefore formulation of a composition to be freeze dried must include consideration not only of the nature and stability characteristics required during the liquid state, both freshly prepared and when reconstituted before use, but the characteristics desired in the final lyophilized cake. Additionally for products to be reconstituted for parenteral usage, consideration must also be given to the pharmacological effects of excipients chosen. In some instances there may even be chemical interaction between the active ingredient and one or more of the excipients during processing. This could, of course, result in reduced potency of the finished product. For all the above reasons, it becomes apparent that selection of a suitable excipient or excipients for a pharmaceutical product containing S-isomer of DRF-1042 is believed to be important.

The formulation, size, shape of the vial, number of vials and type of lyophilizes will control the time required to complete primary drying, which may vary from few hours up to several days. Upon completion of primary drying the shelf temperature is raised to the desired setting to perform secondary drying.

In an embodiment, the invention includes the parameters which are of concern for lyophilized composition, wherein the resulting cake (lyophilized product) was evaluated visually on its physical appearance using as desired criteria: Original shape, no shrinkage or meltback, good coloration, homogeneity, firmness and crystallinity. After the lyophilization process was completed the material remaining in the vial was observed for color appearance, texture, friability, and shrinkage from the original volume. Also each formulation was tested for its moisture loss on drying and its dissolution characteristics, dose uniformity, sterility testing, and so on.

The percent ratio of cake height to vial height may be in the range of from about 20 to 45%.

Reconstitution of the lyophilized composition (which can be stored for an extended period of time at a predetermined temperature) at the desired stage, typically before administration to the patient needs to be reconstituted with an appropriate medium to produce a solution or suspension or dispersion or emulsion. The reconstitution medium may include sterile water, normal saline, water for injection, a pH buffered solution, or 5% dextrose solution (D5W). The reconstitution is usually performed at room temperature, however other temperatures may also be considered. The reconstituted lyophilized composition should passes the USP <788> particulate matter test.

The USP particulate matter test defines the number of foreign particulate matter as observed by optical microscopy. As per USP <788>, the limit for foreign particulate matter having size greater than or equal to 10 microns is 3000, and for particles having size greater than or equal to 25 microns is 300.

It is also envisaged that the solution of the inclusion complex as prepared above could be used as a medicament directly in the form of an oral solution for direct administration or further processed using sterile filtration and aseptic processing to provide sterile solutions for injection. The powder compositions as prepared above may be used as such or may be further converted into different pharmaceutical formulations for administration to patients in need thereof. Such pharmaceutical compositions include for example but are not limited to tablets, capsules, caplets, syrups, solutions, solutions for injection, suspensions, emulsions, dispersions, lyophilized powders and the like. Optionally, the powder compositions may be filled into capsules or into sachets and the like and used directly without further modification by adding a pharmaceutically acceptable excipient. The use of the powder compositions directly as pharmaceutically compositions to be administered to patients in need thereof is also within the scope of the invention.

The compositions described herein may be used in pharmaceutical products and administered through any route which will help in effective delivery of the active ingredient. Routes such as oral route or through parenteral route such as via the intravenous, intramuscular, subcutaneous, intrathecal, intraperiotoneal routes and the like or topically, transdermally, transmucosally.

In another embodiment, there is provided a pharmaceutical formulation for oral administration that includes a therapeutically effective dose of 5(S)-(2′-hydroxyethoxy)-20(S)-CPT in the form of the powder composition. Preferably, the formulation for oral administration includes at least one pharmaceutically acceptable excipient.

Non-limiting examples of excipients include diluents, disintegrants, binders, glidants, antiadherents, lubricants, solvents, pH modifiers, preservatives, antioxidants, colorants, flavouring agents and the like.

Various useful diluents include but are not limited to starches, lactose, mannitol, pearlitol SD 200, cellulose derivatives, confectioner's sugar and the like. Different grades of lactose include but are not limited to lactose monohydrate, lactose DT (direct tableting), lactose anhydrous, Flowlac™ (available from Meggle products), Pharmatose™ (available from DMV) and others. Different grades of starches included but not limited to maize starch, potato starch, rice starch, wheat starch, pregelatinized starch (Commercially available as PCS PC10 from Signet Chemical Corporation) and Starch 1500, Starch 1500 LM grade (low moisture content grade) from Colorcon, fully pregelatinized starch (Commercially available as National 78-1551 from Essex Grain Products) and others. Different cellulose compounds that can be used include crystalline cellulose and powdered cellulose. Examples of crystalline cellulose products include but are not limited to CEOLUS™ KG801, Avicel™ PH 101, PH102, PH301, PH302 and PH-F20, microcrystalline cellulose 114, and microcrystalline cellulose 112. Other useful diluents include but are not limited to carmellose, sugar alcohols such as mannitol, sorbitol and xylitol, calcium carbonate, magnesium carbonate, dibasic calcium phosphate, dicalcium lactose, and tribasic calcium phosphate.

Various useful binders include but are not limited to hydroxypropylcellulose (Klucel™-LF), hydroxypropyl methylcellulose or hypromellose (Methocel™), polyvinylpyrrolidone or povidone (PVP-K25, PVP-K29, PVP-K30, PVP-K90), plasdone S 630 (copovidone), powdered acacia, gelatin, guar gum, carbomer (e.g. carbopol), methylcellulose, polymethacrylates, and starch.

Various useful disintegrants include but are not limited to carmellose calcium (Gotoku Yakuhin Co., Ltd.), carboxy methylstarch sodium (Matsutani Kagaku Co., Ltd., Kimura Sangyo Co., Ltd., etc.), croscarmellose sodium (FMC-Asahi Chemical Industry Co., Ltd.), crospovidone, examples of commercially available crospovidone products including but not limited to crosslinked povidone, Kollidon™ CL [manufactured by BASF (Germany)], Polyplasdone™ XL, XI-10, and INF-10 [manufactured by ISP Inc. (USA)], and low-substituted hydroxypropylcellulose. Examples of low-substituted hydroxypropylcellulose include but are not limited to grades such as LH11, LH21, LH31, LH22, LH32, LH20, LH30, LH32 and LH33 (all manufactured by Shin-Etsu Chemical Co., Ltd.). Other useful disintegrants include sodium starch glycolate, colloidal silicon dioxide, and starch.

Various glidants or antisticking agents, which include but not limited to talc, silica derivatives, colloidal silicon dioxide and the like or mixtures thereof.

Various lubricants that can be used include but are not limited to stearic acid and stearic acid derivatives such as magnesium stearate, calcium stearate, zinc stearate, sucrose esters of fatty acid, polyethylene glycol, talc, sodium stearyl fumarate, zinc stearate, castor oils, waxes.

Various pH modifiers include but are not limited various acids such as hydrochloric acid, phosphoric acid, citric acid, carbonic acid, tartaric acid, fumaric acid, acetic acid etc; various bases such as sodium hydroxide, magnesium hydroxide, calcium hydroxide etc; various salts such as citrates, phosphates, carbonates, tartrates, fumarates, acetates of various alkaline or alkaline earth metals, amino acids, amino acid salts, and meglumine.

Various useful colourants include but are not limited to Food Yellow No. 5, Food Red No. 2, Food Blue No. 2, and the like, food lake colorants, ferric oxide.

The flavoring agents, which can be used in this present invention, are but not limited to natural or synthetic or semi synthetic origin like menthol, fruit flavors, citrus oils, peppermint oil, spearmint oil, oil of wintergreen (Methyl salicylate).

Particularly contemplated are pharmaceutical compositions for oral administration having a defined dissolution profile. Preferred is a formulation which releases 80% or more of 5(S)-(2′-hydroxyethoxy)-20(S)-CPT into solution within 60 minutes after introduction of the pharmaceutical formulation into a biorelevant medium comprising 900 ml of 0.1 N hydrochloric acid at a temperature of 37° C.±0.5° C. in a USP Type II apparatus stirred at 75 rpm. Also preferred is a formulation which releases 80% or more of said 5(S)-(2′-hydroxyethoxy)-20(S)-CPT into solution within 30 minutes after introduction of the pharmaceutical formulation into the biorelevant medium. The modified rates of release are expected to result in improved bioavailability when administered to a patient in need thereof in comparison with a product, which is not a powder composition as per the meaning in the invention.

In a variant, which is particularly contemplated, the pharmaceutical formulation for oral administration is a capsule, the powder composition and the excipient(s) being filled into said capsule. Particularly contemplated are capsule of size 00 (which may be suitable for 25 mg dose) and those of size 3 (which may be suitable for 5 mg dose). In another variant, the formulation is a tablet.

As mentioned above, the amount of 5(S)-(2′-hydroxyethoxy)-20(S)-CPT is determined by particular medical need. In an embodiment, pharmaceutical formulations that include 5(S)-(2′-hydroxyethoxy)-20(S)-CPT in the concentration ranging between about 0.5% to about 50% or about 1% to about 25% by weight of the total composition are separately contemplated. Specifically contemplated are pharmaceutical formulations for oral administration containing from 1 mg to 100 mg of 5(S)-(2′-hydroxyethoxy)-20(S)-CPT. Also contemplated are pharmaceutical formulations for oral administration containing 5 mg, 10 mg, or 25 mg of 5(S)-(2′-hydroxyethoxy)-20(S)-CPT. The amount of the active ingredient in the formulation is adjusted by adjusting the amount of active ingredient included in the powder composition.

The pharmaceutical formulations may be prepared by traditional methods, including direct blending, dry granulation, wet granulation, extrusion and spheronization, fluid bed coating, fluid bed processing and the like without limitation. An example of the preparation process includes:
a) Sifting the powder composition and other pharmaceutically acceptable excipients.
b) Blending the powder compositions with pharmaceutically acceptable excipients
c) Optionally granulating the above blend using aqueous or non-aqueous binder solution or dispersion and drying (e.g., by tray drying, or fluid bed drying)
d) Sizing and sifting the dried granules.
e) Blending the sifted granules with excipients such as lubricants, disintegrants, glidants, and the like.
f) Filling the blend into the capsules or compressing into tablets.

As mentioned, the formulations for parenteral administration (intravenous, intramuscular, subcutaneous, intrathecal, intraperiotoneal) are specifically contemplated. If a formulation is to be administered through parenteral route, the composition is to be rendered sterile prior to administration.

In one embodiment, there is provided is a pharmaceutical formulation for parenteral administration that includes i) a therapeutically effective dose of 5(S)-(2′-hydroxyethoxy)-20(S)-CPT in the form of the powder composition described herein; and ii) a container suitable for a parenteral pharmaceutical product. Preferably, the formulation includes at least one parenterally-acceptable excipient. An example of a parenterally acceptable excipient is a bulking agent, such as sodium chloride or mannitol.

The pharmaceutical formulation of this embodiment is intended for reconstitution with a suitable parenterally acceptable diluent, typically just before administration. After reconstitution, the dosage form is usually administered immediately though it may be acceptable to store for a limited period of time before administration provided the chemical stability and the sterility of the product are not compromised.

Preferably, a container is capable of maintaining a sterile environment. Additionally suitable containers imply appropriateness of size, considering the volume of solution to be held upon reconstitution of the lyophilized composition; and appropriateness of container material, generally USP Type I glass. The stopper means employed, e.g. sterile rubber closures or an equivalent should be understood to be that which provides the afore mentioned seal but which also allows entry for the purpose of introduction of diluent, e.g. sterile water, the reconstitution of the desired solution of S-isomer of DRF-1042. Examples of suitable containers included in the formulation for parenteral administration are a vial, an ampoule and a prefilled syringe.

The containers, including lids and implements, may be made of various materials such as high-density polyethylene (HDPE), low-density polyethylene (LDPE) and or polypropylene and/or glass, and blisters or strips composed of aluminium of high-density polypropylene, polyvinyl chloride, or polyvinyl dichloride.

Molecular sieves may be used to provide a moisture-free environment based on the understanding that one of the drug-related impurities (decarboxylated [5S-(2′-hydroxyethoxy)-20(S)-camptothecin] increases significantly in an environment of higher temperature and humidity.

In another embodiment, there is provided a pharmaceutical formulation for parenteral administration that includes i) a therapeutically effective dose of 5(S)-(2′-hydroxyethoxy)-20(S)-CPT containing less than 5% of 5(R)-(2′-hydroxyethoxy)-20(S)-CPT, and a cyclodextrin in the form of a sterile solution comprising a vehicle suitable for parenteral administration, the 5(S)-(2′-hydroxyethoxy)-20(S)-CPT and said cyclodextrin being dissolved in the diluent; and ii) a container suitable for a parenteral pharmaceutical product. Preferably, 5(S)-(2′-hydroxyethoxy)-20(S)-CPT is substantially free from 5(R)-(2′-hydroxyethoxy)-20(S)-CPT. Preferably, 5(S)-(2′-hydroxyethoxy)-20(S)-CPT is present at a concentration greater than 1 mg/ml. In another variant, (S)-(2′-hydroxyethoxy)-20(S)-CPT is present at a concentration greater than 25 mg/ml.

The pharmaceutical formulation of this embodiment may include at least one parenterally acceptable excipient. Examples of parenterally acceptable excipients include osmolality adjustors, pH adjustors, and preservatives. Other excipients required such as suitable buffers, antioxidants or chelating agents could also be included.

Also contemplated is a kit that includes:

a) a container with the powder composition described herein; and

b) a pharmaceutically acceptable diluent for reconstitution

If desired, a dispenser or other implements may also be included in the kit. Examples of pharmaceutically acceptable diluents include but are not limited to sterile water for injection, dextrose solution, and/or saline solution. A sterile syringe for administration may also be provided for reconstitution and ready administration as part of the kit to enhance the ease of use.

All information about pharmacological activity and utility of S-isomer of DRF 1042 set forth in co-pending and co-assigned U.S. patent application Ser. Nos. 11/753,432 and 11/753,392 is incorporated herein by reference specifically for the purposes stated.

Certain specific aspects and embodiments of the invention will be further described in the following examples, which are provided for purposes of illustration and are not intended to limit the scope of the invention in any manner.

### EXAMPLES

### General Experimental Techniques

Dissolution: Compositions are subjected to dissolution testing as per the following procedure. USP Type II apparatus, at 75 rpm in 900 ml of 0.1N HCl at 37° C.±0.5° C., sampling time 45 minutes.

Samples are analyzed by HPLC using a Chiralcel OD-H 250×4.6 mm column with a 5 μm particle size, at a wavelength of 257 nm using a variable wavelength UV detector, and a mobile phase comprising buffer (0.01 M KH₂PO₄; pH 3.0±0.1): acetonitrile (68:32% v/v), flow rate 1 ml/minute.

For the determination of the R-isomer impurity in the compositions, conditions essentially similar to the ones described above are used. The mobile phase comprises buffer (pH 3.0±0.1): acetonitrile (76:24% v/v).

The location of the impurity peak in the chromatogram is defined by the term “RRT” which as used herein is intended to indicate the relative retention time of the particular impurity against pure DRF-(5S,20S)-1042 (assigned an RRT value of 1) during an HPLC analysis.

Generally, the DRF-(5S,20S)-1042 is extracted from the powder compositions or from the pharmaceutical compositions using a diluent comprising a mixture of methanol, 50% orthophosphoric acid and acetonitrile (30:60:10% v/v) followed by filtration and HPLC analysis as per the procedure described above, after suitable dilution with the mobile phase.

| COMPOUND NAME | RRT |
|---|---|
| DRF-1042 isomer S | 1.0 |
| DRF-1042 isomer R | 0.83 |

For impurities other than the R-isomer the HPLC analysis comprises a Waters HPLC system equipped with a variable wavelength UV detector using symmetry C18, 250 column with a 5 μm particle size, at a wavelength of 257 nm, column temperature of 40° C. The mobile phase comprises

Mobile Phase-A: Phosphate buffer (pH 3.0±0.1) and methanol in the ratio of 90:10% v/v

Mobile Phase-B: Phosphate buffer (pH-3.0±0.1), methanol and acetonitrile in a ratio of 40:30:30% v/v.

### Gradient Program:

| Time (min) | Mobile Phase A (% v/v) | Mobile Phase B (% v/v) |
|---|---|---|
| 0.01 | 60 | 40 |
| 35 | 60 | 40 |
| 40 | 10 | 90 |
| 60 | 10 | 90 |
| 65 | 60 | 40 |
| 70 | 60 | 40 |

The DRF-(5S,20S)-1042 is extracted from the powder compositions or from the pharmaceutical compositions using a diluent comprising methanol, 50% orthophosphoric and acetonitrile (30:60:10% v/v) followed by filtration and HPLC analysis as per the procedure described above, after suitable dilution with the mobile phase B.

| COMPOUND NAME | RRT |
|---|---|
| DRF-1042 isomer S | 1.0 |
| Decarboxylate impurity | 1.45 |
| Dimer | 1.75 |

Throughout all the examples reference has been made to certain abbreviations used for different impurities as follows:

### A=DRF (5R,20S)-1042

B=Decarboxylated impurity.

### C=Dimer Impurity.

D=Total impurities excluding DRF (5R,20S)-1042.

### Example 1

### Physicochemical Properties of DRF-(5S,20S)-1042

DRF-(5S,20S)-1042 was prepared by a process comprising the steps of suspending 5-(2′-hydroxyethoxy)-20(S)-camptothecin in a suitable solvent such as n-butanol or tetrahydrofuran and refluxing over a period of 2-3 hours, reaction mass temperature was slowly lowered to 40-45° C., filtered, washed with n-butanol or tetrahydrofuran and dried.

Table 1 describes the physicochemical characteristics of DRF-(5S,20S)-1042, which is used in the examples below.

TABLE 1

| Parameter | Result |
|---|---|
| Moisture content (% w/w) | 0.2 |
| Bulk density (g/ml) | 0.17 |
| Tapped density (g/ml) | 0.31 |
| Carr Index (%) | 46.5 |
| Hausner ratio | 1.9 |
| Related substances | |
| a) DRF-(5R,20S)-1042 | 1.2% |
| b) Total impurities | 1.9% |
| Particle size for the lot used for the below cited | D₁₀ = 3.6 μm |
| examples | D₅₀ = 10.9 μm |
| | D₉₀ = 34.0 μm |

### Example 2

### Solubility of DRF-(5S,20S)-1042 in Different Media

Excess amounts of DRF-(5S,20S)-1042 were added to different media including water, fasting state simulated intestinal fluid (FaSIF), fed state simulated intestinal fluid (FeSIF), aqueous sodium carbonate solution (0.1M, pH12.6), aqueous sodium hydroxide solution (0.1M, pH 12.73) and the suspensions were shaken at room temperature for 24 hours at 200 rpm in a mechanical shaker water bath till no further drug went into solution when checked visually. The suspensions were filtered through a 0.22 μm membrane filter (supplied by Millipore) and the content of DRF-(5S,20S)-1042 was quantified by using the HPLC method described above. The data is described in table 2.

TABLE 2

| Medium | Solubility (μg/mL) |
|---|---|
| Water | [symbol] 4 |
| FaSIF | [symbol] 4 |
| FeSIF | ~41 |
| Sodium carbonate solution (0.1M, pH12.6) | 4161.6 |
| Sodium hydroxide solution (0.1M, pH 12.73) | 6672.2 |

[symbol]

indicates data missing or illegible when filed

The above data demonstrate the poor solubility of DRF-(5S,20S)-1042 in various media and the increasing solubility in alkaline conditions. They also demonstrate the need for a significant improvement in the solubility properties of the compound in order to formulate into a pharmaceutical dosage form for oral or parenteral delivery.

### Example 3

### Phase Solubility Study of S-Isomer of DRF 1042 in Aqueous Solution of HPBCD without Using an Alkalizer

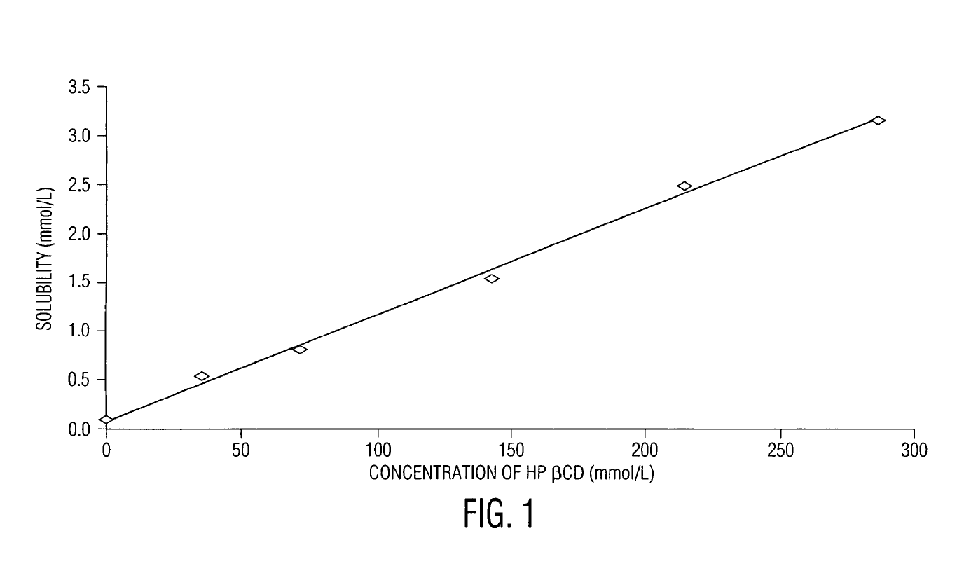

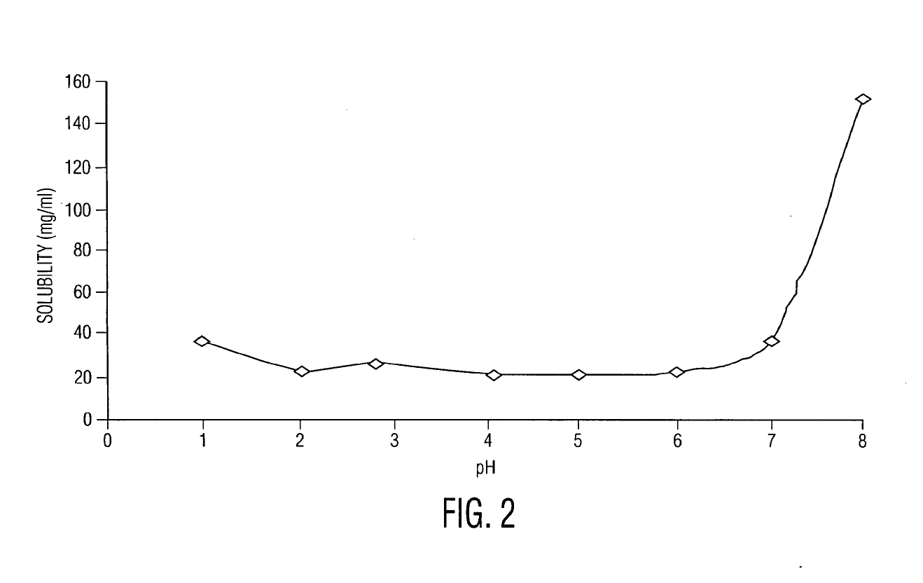

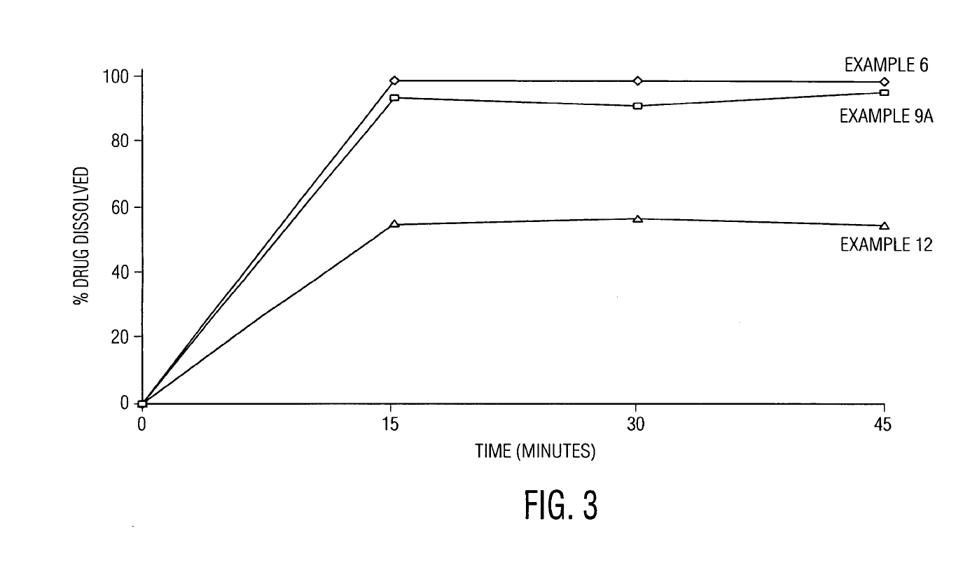

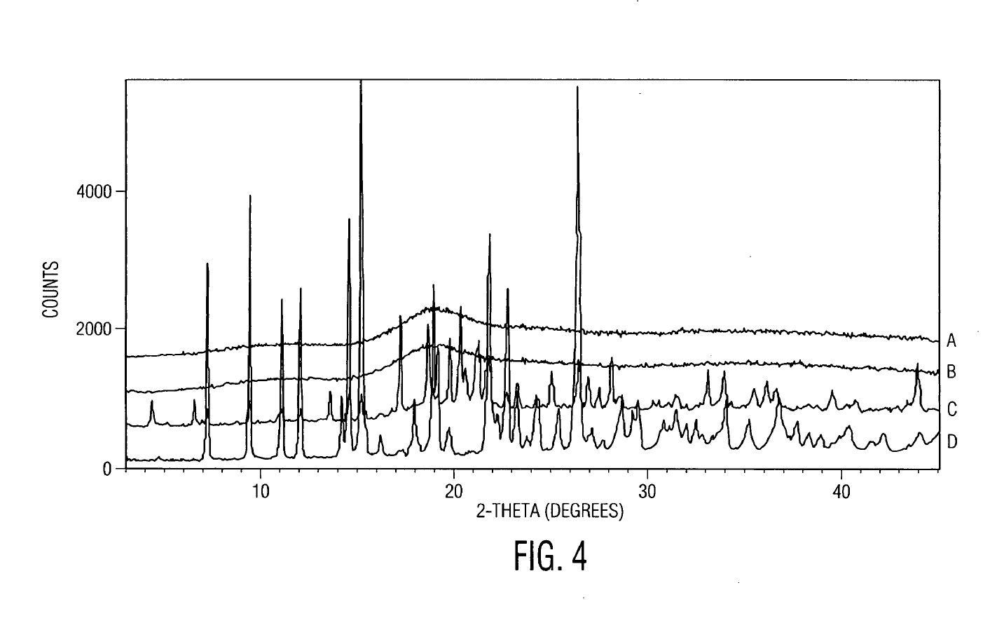

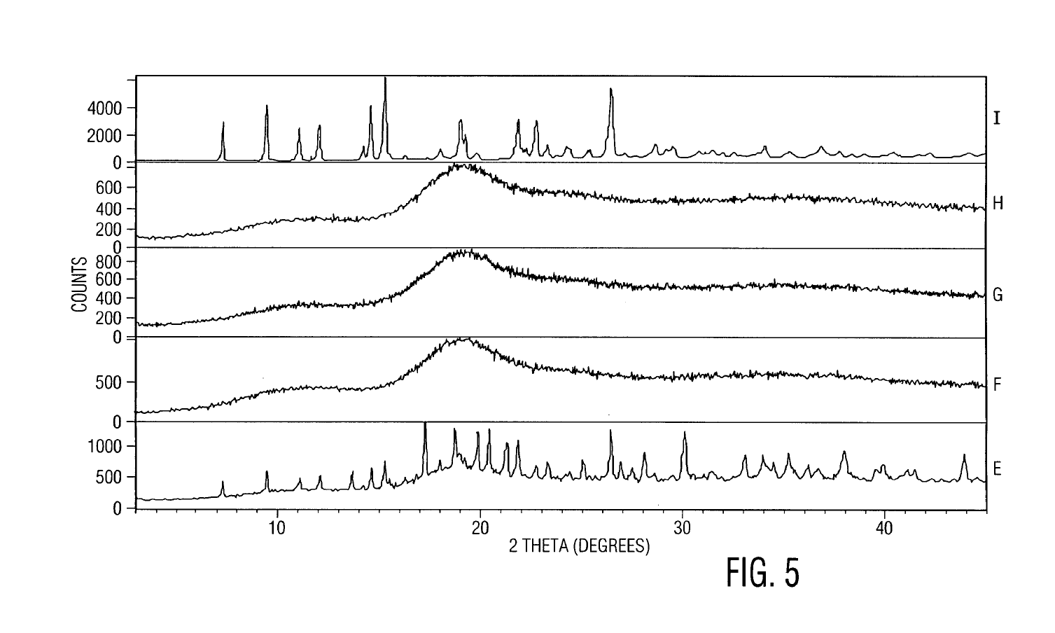

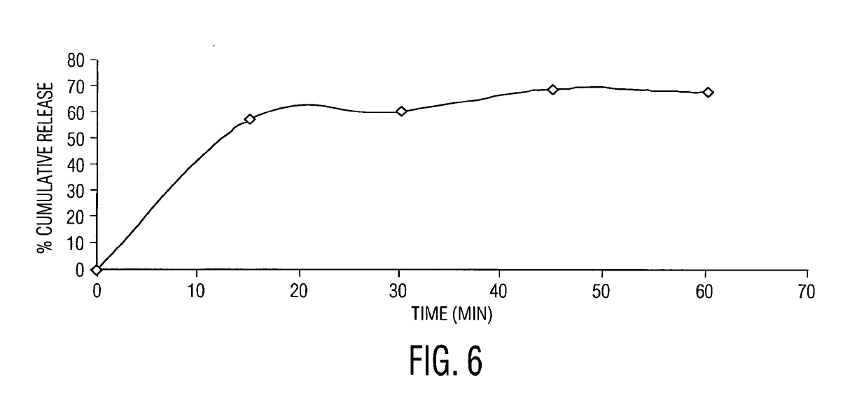

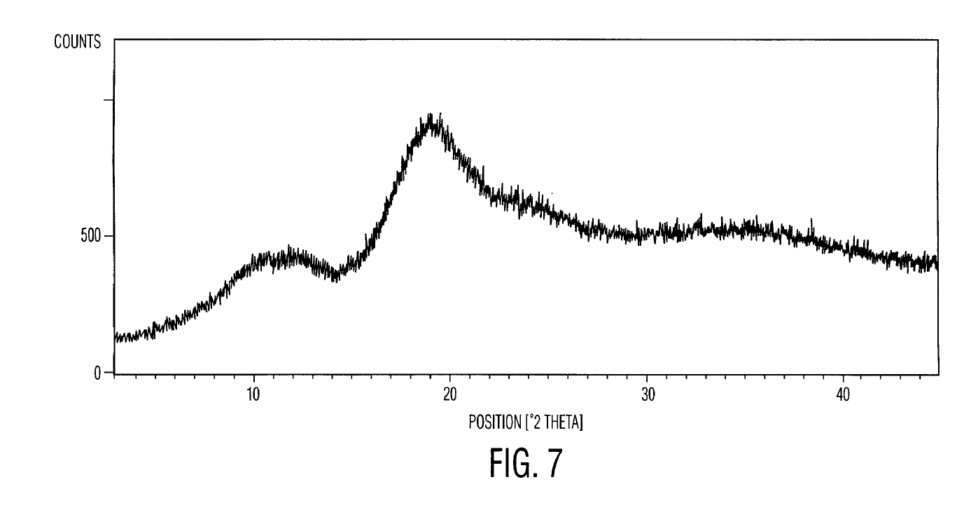

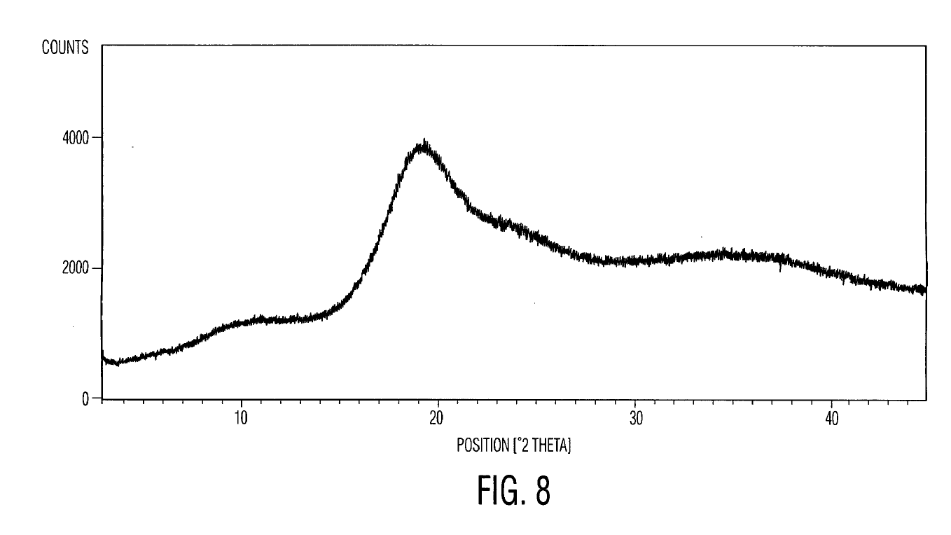

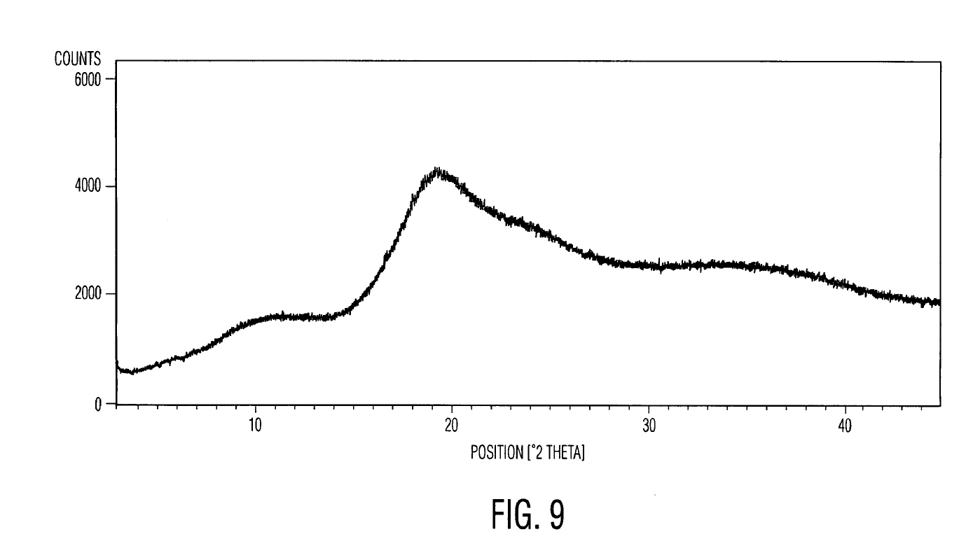

Excess amounts of DRF-(5S,20S)-1042 were added to aqueous solutions of HPBCD with concentrations ranging from 0-40% w/v and the suspensions were shaken at 200 rpm and 25° C. in an incubator shaker, till no further DRF-(5S,20S)-1042 went into solution. The solutions were filtered through a 0.22 μm membrane filter and subjected to analysis by HPLC by a process described above. The results are described in Table 3 and in FIG. 1.

TABLE 3

| HPBCD concentration (% w/v) | Solubility (mg/ml) |
|---|---|
| 0 | 0.02 |
| 10 | 0.21 |
| 20 | 0.62 |
| 40 | 1.29 |

The data demonstrate an increase in aqueous solubility of DRF-(5S,20S)-1042 of over 50-fold when compared with water alone.

### Example 4

### Solution Stability of DRF-(5S,20S)-1042 in Different Alkaline Solutions

To a 10 mg/ml dispersion of DRF-(5S,20S)-1042 in purified water was added an alkalizer selected from sodium hydroxide, meglumine or arginine in a ratio of 1:2 of drug and alkalizer. About half of the volume of each alkalized solution was neutralized to a pH of 7.5 using orthophosphoric acid to form the ‘neutralized sample’ while the remaining half of the solution was retained as the ‘as is’ sample with a pH greater than 10.5. Both sets of samples were analyzed for the impurities generated for DRF-(5S,20S)-1042 in the solution initially as well as after storing for 24 hours at room temperature (RT), by the HPLC procedure described above. The data is tabulated in Table 4.

TABLE 4

| | Initial | | | | 24 hours RT | | | |
|---|---|---|---|---|---|---|---|---|
| Alkalizer | A | B | C | D | A | B | C | D |
| NaOH as is | 75.40 | 0.02 | 0.35 | 1.81 | 101.79 | 0.02 | 0.48 | 1.95 |
| NaOH | 56.75 | 0.02 | 0.38 | 1.81 | 56.26 | 0.02 | 0.37 | 2.22 |
| neutralized | | | | | | | | |
| Meglumine | 2.77 | 0.21 | 12.04 | 21.15 | 2.30 | 0.33 | 7.17* | 11.1 |
| as is | | | | | | | | |
| Meglumine | 1.05 | 0.03 | 0.54 | 1.49 | 2.57 | 0.33 | 1.47 | 5.02 |
| neutralized | | | | | | | | |
| Arginine | 3.45 | 0.06 | 0.88 | 2.50 | 5.22 | 0.19 | 0.95 | 3.23 |
| as is | | | | | | | | |
| Arginine/ | 3.38 | 0.04 | 0.9 | 2.64 | 3.26 | 0.18 | 0.93 | 3.11 |
| neutralized | | | | | | | | |

The data demonstrate the significant conversion of the S-isomer into the R-isomer and the incomplete conversion to the S-isomer upon neutralization. The data also demonstrate the importance of the solution pH during processing and for the final formulation to ensure product stability.

### Example 5

### Powder Composition of DRF-(5S,20S)-1042 with HPBCD

| Ingredient | mg/ml |
|---|---|
| DRF-(5S,20S)-1042 | 10 |
| HPBCD | 75 |
| Water | 1 ml |

DRF-(5S,20S)-1042 and HPBCD were mixed together and this physical mixture was sifted through #40 ASTM mesh sieve. Purified water was added to the above physical mixture and sonicated for 1 hour. It was observed that even after sonication, a clear solution was not formed. The drug remained in suspension even after heating at 60° C. for 30 minutes and under stirring for 1 hour.

This shows that plain HPBCD was not sufficient to solubilize 5(S)-CPT.

### Example 6

### Composition of DRF-(5S,20S)-1042 with HPBCD and Sodium Lauryl Sulphate

| Ingredient | mg/capsule |
|---|---|
| DRF-(5S,20S)-1042 | 10 |
| HPBCD | 65 |
| Sodium lauryl sulphate | 20 |
| Water* | 1 ml |

*Evaporates during drying

DRF-(5S,20S)-1042, HPBCD and sodium lauryl sulfate were mixed together and sifted through a #40 ASTM mesh sieve. To this mixture purified water was added to form a dispersion. This dispersion was then sonicated for 1 hour to obtain a clear solution, which was subsequently filtered through a 0.22 μm membrane filter and analyzed by the HPLC method described above after suitable dilution.

The above experiment, demonstrates that using a combination of sodium lauryl sulphate along with HPBCD allows the solubilization of DRF-(5S,20S)-1042.

### Example 7

### Powder Composition of DRF-(5S,20S)-1042 with HPBCD, Sodium Lauryl Sulphate and Mannitol

| Ingredients | mg/capsule |
|---|---|
| DRF-(5S,20S)-1042 | 5 |
| HPβCD | 37.5 |
| Sodium Lauryl Sulphate | 5 |
| Mannitol | 5 |
| Purified water* | 0.5 ml |

*Evaporates during drying

### Manufacturing Process:

The inclusion complex solution was prepared essentially as per the process described in the previous example (Example 6) except that mannitol has been included in the physical mixture of DRF-(5S,20S)-1042, HPBCD and sodium lauryl sulfate. The clear solution was further subjected to spray drying using a Buchi spray drier at an inlet temperature of 140±5° C., an outlet temperature of 80±2° C., an aspiration rate of 110-130 mm water column and at a Spray pump rate of 20 rpm to obtain a dry powder composition. The spray dried powder composition was subsequently vacuum dried to a final moisture content below 8% as measured by Karl-Fischer titration.

The dry powder composition was filled into size 3 hard gelatin capsules to prepare the pharmaceutical formulation of the invention, packed in sealed amber colored glass vials and kept for 24 hours at 60° C. The samples were analyzed for impurities by using HPLC as per the procedures described above. The data is tabulated in table 5.

TABLE 5

Drug-related
Impurities
(% Peak Area)

| Impurity | Initial | 24 Hours |
|---|---|---|
| DRF-(5R,20S)-1042 | 0.60 | 18.07 |
| Decarboxylated | 0.05 | 0.15 |
| Total Impurities | 1.02 | 18.89 |

### Example 8

### Powder Composition of DRF-(5S,20S)-1042 with Sodium Carbonate

| Ingredient | mg/ml |
|---|---|
| DRF-(5S,20S)-1042 | 10 |
| HPBCD | 75 |
| Mannitol | 10 |
| Sodium carbonate | 10 |
| Purified water* | 1 ml |

*Evaporates during drying

### Manufacturing Process:

The inclusion complex solution was prepared essentially as per the process described in the Example 6. About half of the volume of the inclusion complex solution was neutralized to pH 7.4 using orthophosphoric acid and the remaining half of the solution was retained as ‘as-is’ (pH>11). Both sets of samples (neutralized and unneutralized) were analyzed initially and at the end of 24 hours by an HPLC procedure described above. The solutions were filled in amber colored glass vials, sealed and exposed to 60° C. for 24 hours. The data were tabulated in Table 6.

TABLE 6

| Related impurities | Neutralized | Unneutralized |
|---|---|---|
| DRF (5R,20S)-1042 | 3.69 | 35.9 |
| Decarboxylated | 0.26 | 0.2 |
| Individual maximum impurity | 0.37 | 0.65 |
| Total impurities | 4.6 | 36.7 |

This example demonstrates that DRF (5R,20S)-1042 degrades rapidly in alkaline conditions.

### Example 9A-9C

### Powder Composition for 5/25 Mg Capsule with Sodium Carbonate as the Complexation Enhancer and Acetonitrile and Purified Water as the Solvent Medium

| | mg/Capsule | | | | | |
|---|---|---|---|---|---|---|
| Ingredients | Example 9A | | Example 9B | | Example 9C | |
| DRF-(5S,20S)-1042 | 5 | | 5 | | 25 | |
| HPBCD | 37.5 | | 37.5 | | 187.5 | |
| Mannitol | 3.75 | | 3.75 | | 18.75 | |
| Sodium carbonate | 0.5 | | 0.625 | | 3.125 | |
| Acetonitrile | 12 | ml | 3 | ml | 15 | ml |
| Purified Water | 5 | ml | 0.3 | ml | 2.75 | ml |

### Manufacturing Process

DRF-(5S,20S)-1042 was dissolved in acetonitrile at 75° C. in a reactor vessel to form the organic phase. HPBCD, mannitol and sodium carbonate were added to purified water and stirred until clear to form the aqueous phase. The organic phase from step 1 was added to the aqueous phase of step 2 with continuous stirring in a reactor vessel at about 50° C. to allow complexation. Acetonitrile was removed under vacuum using a rotavaporator. Concentrated complex solution was filtered using a 0.22 μm membrane filter and subjected to spray drying to obtain a dry powder composition.

The process parameters used for spray drying were:

inlet temperature: 140±5° C.,
outlet temperature: 85±2° C.;
aspiration rate: 110-130 mm WC (water column),
spray pump rate: 20 RPM.
The spray dried drug complex was subsequently vacuum dried to obtain a final moisture content below 8% as determined using Karl Fischer titration.

The dry complex powder of Example 9A was filled into size 3 hard gelatin capsules and packed in sealed amber coloured glass vials and kept for 24 hours at 60° C. The capsules were analyzed for impurities by using an HPLC procedure as described above. The data is tabulated in Table 7.

TABLE 7

Drug-related
Impurities (%
Peak Area)

| Impurity | Initial | 24 Hours |
|---|---|---|
| DRF-(5R,20S)-1042 | 0.7 | 35.9 |
| Decarboxylated | 0.06 | 0.2 |
| Total Impurities | 1.26 | 36.7 |

The capsules of Example 9B were charged for stability for about 3 months at different temperature conditions such as 2-8° C., 25° C. and the samples were analyzed by an HPLC procedure described above. The results are tabulated in below Table 8.

TABLE 8

| | % Peak Area | | | | | | | |
|---|---|---|---|---|---|---|---|---|
| | 2-8° C. | | | | 25° C. | | | |
| Impurities | Initial | 1 M | 2 M | 3 M | Initial | 1 M | 2 M | 3 M |
| A | 0.74 | 0.8 | 0.86 | 0.85 | 0.74 | 0.91 | 1.07 | 1.21 |
| B | 0.23 | 0.25 | 0.28 | 0.28 | 0.23 | 0.34 | 0.47 | 0.55 |
| C | 0.31 | 0.34 | 0.29 | 0.32 | 0.31 | 0.34 | 0.31 | 0.33 |
| D | 1.53 | 1.55 | 1.61 | 1.59 | 1.48 | 1.78 | 2.08 | 2.31 |

### Pharmaceutical Formulations Comprising the Powder Composition of Example 9C

| Ingredient | mg/capsule |
|---|---|
| Powder composition | 242.64 |
| (Example 9C) | |
| Lactose monohydrate | 252.86 |
| (Lactose DCL-21) | |
| Magnesium stearate | 4.86 |

### Manufacturing Process

The powder composition obtained in Example 9C was mixed with the specified amount of lactose DCL-21 and sifted through a #30 ASTM mesh sieve and the mixture was blended for 10 minutes in a blender. Magnesium stearate was added to above blend and blended for another 10 minutes. The lubricated blend was filled into Size “00” capsules.

### Example 10

### Pharmaceutical Formulations of Powder Composition of DRF-(5S,20S)-1042 with Sodium Carbonate as Complexation Enhancer

| Ingredient | mg/ml | |
|---|---|---|
| DRF-(5S,20S)-1042 | 10 | |
| HPBCD | 75 | |
| Mannitol | 10 | |
| Sodium lauryl sulphate | 1 | |
| Sodium carbonate | 12 | |
| Purified water* | 1 | ml |

### Manufacturing Process

DRF-(5S,20S)-1042, HPBCD, sodium carbonate and sodium lauryl sulfate were mixed together and sifted through a #40 ASTM mesh sieve. To this mixture purified water was added then to form a dispersion. This dispersion was then sonicated for 1 hour to obtain a clear solution, which was subsequently filtered through a 0.22 μm membrane filter and the pH was adjusted to about 7 with orthophosphoric acid.

### Spray Drying

The drug complex in solution was subsequently spray dried to obtain dry powder complex. The process parameters used for spray drying such as inlet Temperature: 100±5° C., outlet Temperature as 65±2° C.; aspiration Rate 110-130 mm WC (water column), Spray pump rate about 20 RPM.

### Pharmaceutical Formulation of the Powder Composition.

| Ingredient | mg/capsule |
|---|---|
| Powder composition | 260 |
| Dicalcium phosphate | 120 |
| Dicalcium lactose-21 | 120 |
| Pregelatinized starch (Starch | 88 |
| 1500 LM) | |
| Colloidal silicon dioxide | 6 |
| Talc | 3 |
| Magnesium stearate | 3 |

### Manufacturing Process

The powder composition was mixed with the specified amount of dicalcium lactose, dicalcium phosphate, starch 1500, colloidal silicon dioxide and sifted through a #30 ASTM mesh sieve and the mixture was blended for 10 minutes. Talc and magnesium stearate sifted through an ASTM #80 mesh were added to the above blend and blended for another 5 minutes. The lubricated blend was filled into size “00” capsules.

### Examples 11-12

### Pharmaceutical Formulations of the Powder Compositions of DRF-(5S,20S)-1042 with Sodium Lauryl Sulphate and Meglumine (Example 11) and Sodium Carbonate (Example 12) as Complexation Enhancer

### Powder Composition:

mg/ml

| Ingredient | Example 11 | | Example 12 | |
|---|---|---|---|---|
| DRF-(5S,20S)-1042 | 10 | | 10 | |
| HPBCD | 75 | | 75 | |
| Mannitol | 10 | | 10 | |
| Sodium lauryl sulphate | 1 | | 1 | |
| Meglumine | 8 | | — | |
| Sodium carbonate | — | | 8 | |
| Purified water* | 1 | ml | 1 | ml |

*Evaporates during drying

### Manufacturing Process

The powder compositions were prepared essentially as per the process described in Example 7 except that meglumine (Example 11) and sodium carbonate (Example 12) are included in the physical mixture comprising DRF-(5S,20S)-1042, HPBCD, mannitol and sodium lauryl sulfate.

### Pharmaceutical Formulations Comprising the Powder Compositions.

| | |
|---|---|
| Ingredient | mg/capsule |

Example 11 and Example 12

| | |
|---|---|
| Powder composition | 260 |
| Dicalcium phosphate (DCP) | 120 |
| Dicalcium lactose-21(DCL-21) | 120 |
| Pregelatinized starch (Starch | 88 |
| 1500 LM) | |
| Colloidal silicon dioxide | 6 |
| Talc | 3 |
| Magnesium stearate | 3 |

### Manufacturing Process

Powder composition, DCP, DCL-21, starch 1500 LM, colloidal silicon dioxide were sifted through a #40 ASTM mesh sieve and talc and magnesium stearate through #80 ASTM mesh sieve. All the sifted materials were blended together in a non shear blender for about 15 minutes. The blend was filled into size “00” hard gelatin capsule shells with a fill weight of 600 mg using a capsule filling machine and these capsules were packed in 40 cc HDPE (High density polyethylene) bottles with molecular sieves as desiccant, rayon filler cotton plug and finally bottle is induction sealed using CRC cap.

### Examples 13-14

### Pharmaceutical Formulations of Powder Compositions of DRF-(5S,20S)-1042 with Different Concentrations of L-Arginine as Complexation Enhancer

### Powder Compositions

mg/ml

| Ingredient | Example 13 | | Example 14 | |
|---|---|---|---|---|
| DRF-(5S,20S)-1042 | 10 | | 10 | |
| HPBCD | 75 | | 75 | |
| Mannitol | 10 | | 10 | |
| Sodium lauryl sulphate | 1 | | 1 | |
| L-arginine | 8 | | 20 | |
| Purified water* | 1 | ml | 1 | ml |

*Evaporates during drying

### Manufacturing Process

The powder compositions were prepared essentially as per the process described in Example 7 except that L-arginine was included as a complexation enhancer in the physical mixture of DRF-(5S,20S)-1042, HPBCD, mannitol, sodium lauryl sulphate. Pharmaceutical formulations: Composition and manufacturing process was the same as described in Example 11.

The powder composition of Example 13 was filled in amber coloured glass vials and sealed. Both initial sample and samples exposed at 25° C./60% RH (relative humidity), 30° C./65% RH, 40° C./75% RH for period of 3 days were analyzed for impurities by using HPLC as per the process described above. The data has been tabulated in Table 10.

TABLE 10

| | Related substances | | | |
|---|---|---|---|---|
| | DRF (5R, | | | Total |
| Time interval | 20S)-1042 | Decarboxylated | Dimer | impurities |
| Initial | 0.48 | 0.08 | 0.36 | 1.2 |
| 3 days 25° C./60% | 0.53 | 0.18 | 0.34 | 1.37 |
| 3 days 30° C./65% | 0.72 | 0.13 | 0.45 | 1.63 |
| 3 days 40° C./75% | 0.48 | 0.22 | 0.3 | 1.36 |

The powder composition and formulation blend along with their placebo have been characterized for their physical parameters and the data has been tabulated in table 11.

TABLE 11

| | |
|---|---|
| Powder | Formulation |
| composition | Blend |

| Parameter | Placebo | Ex 11 | Placebo | Ex 11 |
|---|---|---|---|---|
| Bulk density (g/ml) | 0.47 | 0.24 | 0.56 | 0.32 |
| Tapped density (g/ml) | 0.67 | 0.36 | 0.70 | 0.56 |
| Compressibility index | 23.61 | 35.19 | 19.10 | 42.9 |
| (%) | | | | |
| Hausner ratio | 1.31 | 1.54 | 1.24 | 1.75 |
| Angle of repose | — | — | — | 34° |

The data demonstrate that the blend has acceptable flow properties for automated capsule filling.

Solution stability of the formulation blend at 40 mg/ml was studied for 48 hours at 2-8° C. and −10° C. and the data has been tabulated in table 12.

TABLE 12

| | Assay (mg/ml) | | |
|---|---|---|---|
| Condition | Initial | 48 hours | 1 week |
| 2-8° C. | 38.6 | 38.3 | NA |
| −10° C. | 38.6 | 38.7 | 38.9 |

The data demonstrate that the blend in solution form was stable and does not precipitate out of the solution even at a very low temperature such as −10° C.

The capsules have been exposed at 25° C./60% RH, 30° C./65% RH and 40° C./75% RH for a period of 3 months and data has been tabulated in Table 13.

TABLE 13

| | % Peak Area | | | | | | | | | | |
|---|---|---|---|---|---|---|---|---|---|---|---|
| Impurity/ | | | 25° C./60% RH | | | 30° C./65% RH | | | 40° C./75% RH | | |
| Isomer | E* | Initial | 1 M | 2 M | 3 M | 1 M | 2 M | 3 M | 1 M | 2 M | 3 M |
| A | 1.11 | 1.19 | 1.18 | 1.19 | 1.53 | 1.26 | 1.31 | 1.31 | 1.49 | 1.51 | 1.4 |
| B | 0.02 | 0.11 | 0.18 | 0.42 | 0.32 | 0.3 | 0.43 | 0.51 | 0.73 | 0.9 | 1.03 |
| C | 0.35 | 0.32 | 0.33 | 0.33 | 0.31 | 0.3 | 0.32 | 0.32 | 0.29 | 0.31 | 0.27 |
| D | 0.96 | 1.07 | 1.03 | 1.19 | 1.53 | 1.23 | 1.53 | 1.97 | 1.80 | 2.06 | 2.57 |

*S-isomer

### Example 15

### Pharmaceutical Formulations of Powder Compositions of DRF-(5S,20S)-1042 with Lactose Monohydrate Used in the Formulation

### Powder Composition:

| Ingredient | mg/capsule | |
|---|---|---|
| DRF-(5S,20S)-1042 | 25 | |
| HPBCD | 187.5 | |
| Mannitol (Pearlitol SD 200) | 25 | |
| Sodium lauryl sulphate | 2.5 | |
| L-arginine | 50 | |
| Purified water* | 2.5 | ml |

*Evaporates during drying

### Manufacturing Process

DRF-(5S,20S)-1042, HPβCD, mannitol, L-arginine and sodium lauryl sulphate were added to purified water to form a dispersion so that final concentration of DRF-(5S,20S)-1042 in the dispersion is 10 mg/ml. The dispersion was stirred using an overhead stirrer at a speed of 100 rpm until a clear solution was obtained with sonication if required. The solution was filtered through a 0.45 μm membrane filter and the pH of the complex solution was adjusted to 7-7.5 using 0.1 N aqueous orthophosphoric acid. The drug complex solution was subjected to spray drying using a spray drier at an inlet temperature of 100° C.±5° C. an outlet temperature of 65° C.±5° C. an aspiration rate more than 1600 to maintain negative pressure and at a Spray pump rate of about 20 rpm to obtain a dry powder composition. The spray dried powder composition was subsequently vacuum dried till the final moisture content was below 8% w/w as measured by Karl-Fischer titration.

### Pharmaceutical Formulation Comprising the Powder Compositions.

| Ingredient | mg/capsule |
|---|---|
| Powder composition | 290 |
| Dicalcium phosphate (DCP) | 74 |
| Lactose monohydrate | 150 |
| Pregelatinized starch (Starch | 74 |
| 1500 LM) | |
| Colloidal silicon dioxide | 6 |
| Talc | 3 |
| Magnesium stearate | 3 |

### Manufacturing Process

Powder composition, DCP, lactose monohydrate, starch 1500 LM, colloidal silicon dioxide were sifted through a #40 ASTM mesh sieve and talc and magnesium stearate through #80 ASTM mesh sieve. All the sifted materials were blended together in a non shear blender for about 15 minutes. The blend was filled into size “00” hard gelatin capsule shells with a fill weight of 600 mg using capsule filling machine and these capsules were packed in 40 cc HDPE bottle with molecular sieves as desiccant, rayon filler cotton plug and finally bottle is induction sealed using CRC cap.

The capsules packed in HDPE containers were exposed to different stability conditions such as 40° C./75% RH, 30° C./65% RH, 25° C./60% RH, 2-8° C. for about 6-12 months. The data are tabulated in the below table 14.

TABLE 14

| | % Peak Area | | | | | | | | | |
|---|---|---|---|---|---|---|---|---|---|---|
| | | 40° C./75% RH | | | 30° C./65% RH | | | 25° C./60% RH | | |
| Impurity | Initial | 2 M | 3 M | 6 M | 2 M | 3 M | 6 M | 3 M | 6 M | 12 M |
| A | 1.19 | 1.51 | 1.4 | 2.26 | 1.31 | 1.31 | 1.57 | 1.19 | 1.41 | 1.41 |
| B | 0.11 | 0.9 | 1.03 | 2.31 | 0.43 | 0.51 | 0.84 | 0.32 | 0.52 | 0.69 |
| C | 0.32 | 0.31 | 0.27 | 0.34 | 0.32 | 0.32 | 0.35 | 0.31 | 0.31 | 0.31 |
| D | 1.08 | 2.03 | 2.57 | 3.96 | 1.53 | 1.97 | 2.12 | 1.53 | 1.9 | 1.97 |
| Dissolution | 96 | 96 | 95 | 98 | 97 | 96 | 998 | 99 | 100 | NP |
| Assay | 24.8 | 23.6 | 23.9 | 23.9 | 24.7 | 24.3 | 24.3 | 24.9 | 24.9 | 24.4 |

NP—Not performed

### Example 17-18

### Pharmaceutical Formulations of In Situ Complexation

| | |
|---|---|
| Example 17 | Example 18 |

| | |
|---|---|
| Ingredient | w/w(g) |

| | | |
|---|---|---|
| DRF (5R,20S)-1042 | 1 | 1 |
| HPβCD | 7.5 | 7.5 |
| Meglumine | 1 | 1 |
| Poloxamer | — | 1 |
| Silicified microcrystalline | — | 1 |
| cellulose | | |

### Manufacturing Process

1) DRF (5R,20S)-1042 and HPβCD, meglumine alone or in combination with poloxamer, silicified microcrystalline cellulose (Example 18), milled together in ball mill for about 6 hours.

The composition from Example 17 was subjected to dissolution in 500 ml of water or 0.1 N HCl in a, USP Type II apparatus

TABLE 17

| Time | | |
|---|---|---|
| (min) | Water | 0.1N HCl |
| 15 | 104 | 55 |
| 30 | 105 | 58 |
| 45 | 105 | 56 |
| 60 | 104 | 58 |

### Example 19

### Pharmaceutical Compositions of DRF-(5S,20S)-1042 Using Granulation Technique. (In Situ Complex)

| Ingredients | mg/capsule |
|---|---|
| DRF-(5S,20S)-1042 | 40 |
| HPβCD | 300 |
| Mannitol | 40 |
| Sodium carbonate | 20 |
| Croscarmellose sodium | 5 |
| PVP K-30 | 5 |
| Sodium lauryl sulphate | 2.5 |
| Purified water | qs |

1) DRF-(5S,20S)-1042 and HPβCD, sodium carbonate were taken into a mortar and triturated to produce intimate contact.
2) Mannitol, croscarmellose sodium and PVP K-30 were added to the above mixture and dry mixed.
3) Sodium lauryl sulphate was dissolved in purified water and the above physical mixture was granulated with this solution to form a coherent mass.
4) The granules were dried in a tray dryer.
5) The dried granules were sifted through an ASTM 40# mesh screen.
6) The above sifted granules were filled into size 00 HG capsules and subjected to dissolution in water and SGF (0.1 N HCl), type II apparatus.

TABLE 18

| Time (min) | Water | 0.1N HCl |
|---|---|---|
| 15 | 82 | 43 |
| 30 | 83 | 45 |
| 45 | 83 | 47 |
| 60 | 84 | 48 |

### Example 20

### Powder Compositions of DRF-(5S,20S)-1042 for Parenteral Administration

| Ingredient | % w/v |
|---|---|
| DRF-(5S,20S)-1042 | 0.5 |
| HPβCD | 3.75 |
| L-arginine | 1 |
| Mannitol | 0.5 |
| Water (MilliQ water) | Q.S to 100 |

HPβCD, L-arginine, and mannitol were added to MilliQ water with continuous stirring and then DRF-(5S,20S)-1042 was added to form a dispersion. This dispersion when agitated continuously for about two and half hours at 550 rpm formed a clear solution. pH was adjusted to 7.62 using 1M orthophosphoric acid and then the solution was subjected to filtration wherein the solution was filtered through 47 mm pre filter (glass fiber filters, Supplier Millipore AP 20), 0.45μ PVDF membrane filter (Supplier Millipore) and 0.22μ PVDF membrane filter (Supplier Millipore) using vacuum filtration assembly.

10 mL of the filtrate was filled into 20 mL capacity USP Type I clear tubular glass vial, which are stoppered partially using 20 mm Lyotech (make West pharma) single leg rubber stopper.
These vials were subjected to lyophilization as per the below mentioned details:

Lyophilization Cycle:

| | | Temperature | | Pressure |
|---|---|---|---|---|
| Step No | Hold/Rate | (° C.) | Time (Min) | (milliTorr) |
| Freezing | | | | |
| Step 1 | Hold | 25° C. | 10 | — |
| Step 2 | Rate | 5° C. | 20 | — |
| Step 3 | Hold | 5° C. | 150 | — |
| Step 4 | Rate | −5° C. | 180 | — |
| Step 5 | Hold | −5° C. | 60 | — |
| Step 6 | Rate | −20° C. | 90 | — |
| Step 7 | Hold | −20° C. | 60 | — |
| Step 8 | Rate | −40° C. | 30 | — |
| Step 9 | Hold | −40° C. | 120 | — |
| Primary drying | | | | |
| Step 1 | Hold | −40° C. | 240 | 200 |
| Step 2 | Rate | −35° C. | 30 | 200 |
| Step 3 | Hold | −35° C. | 240 | 200 |
| Step 4 | Rate | −25° C. | 30 | 200 |
| Step 5 | Hold | −25° C. | 360 | 200 |
| Step 6 | Rate | −15° C. | 30 | 200 |
| Step 7 | Hold | −15° C. | 360 | 200 |
| Step 8 | Rate | −5° C. | 30 | 200 |
| Step 9 | Hold | −5° C. | 360 | 200 |
| Step 10 | Rate | 0° C. | 30 | 150 |
| Step 11 | Hold | 0° C. | 60 | 150 |
| Step 12 | Rate | 10° C. | 30 | 150 |
| Step 13 | Hold | 10° C. | 60 | 150 |
| Step 14 | Rate | 20° C. | 30 | 150 |
| Step 15 | Hold | 20° C. | 120 | 150 |
| Step 16 | Hold | 25° C. | 150 | 150 |
| Secondary drying | | | | |
| Step1 | Hold | 30° C. | 720 | 50 |

Freezing time: 12 hours
Primary drying: 36 hours
Secondary drying: 12 hours.

After completion of lyophilization cycle the vials were sealed using 20 mm aluminum flip-off seals. The resulting lyophilized product was tested for various parameters as per Table 19.

TABLE 19

| Parameter | Result |
|---|---|
| Moisture content by KF | 1.93% w/w |
| pH | 7.53 |
| Assay | 52.82 mg/vial |
| R-isomer | 1.14% |
| Decarboxylated impurity | 0.2% |
| Dimer impurity | 0.33% |
| Total impurities | 1.28% |

Examples 1, 2, 3, 4, 5, 6, 7, 8, and 9 of co-pending and co-assigned U.S. patent application Ser. No. 11/753,432 are expressly incorporated by reference. In addition, these Examples are reproduced below.

### Example 21

This example shows the improved topoisomerase I inhibition activity of 5(S)-(2′-hydroxyethoxy)-20(S)-camptothecin as compared against the 5(RS)-(2′-hydroxyethoxy)-20(S)-camptothecin diastereoisomeric mixture and against the 5(R)-(2′-hydroxyethoxy)-20(S)-camptothecin diastereoisomer.

### Preparation of 5(S)-(2′-hydroxyethoxy)-20(S)-camptothecin, 5(R)-(2′-hydroxyethoxy)-20(S)-camptothecin and 5(RS)-(2′-hydroxyethoxy)-20(S)-camptothecin

A diastereoisomeric mixture of 5(RS)-(2′-hydroxyethoxy)-20(S)-camptothecin (75 grams) prepared as described in Example 26 of U.S. Pat. No. 6,177,439, was suspended in n-butanol (about 600 ml) and refluxed over a period of about 2-3 hours. The reaction mass temperature was reduced over a period of about 1 hour to about 40-50.degree. C., and the solid material obtained was filtered, washed with n-butanol (about 15-20 ml) and dried under vacuum at about 50-55.degree. C. to yield solid 5(S)-(2′-hydroxyethoxy)-20(S)-camptothecin substantially free of 5(R)-(2′-hydroxyethoxy)-20(S)-camptothecin. The product was further enriched to yield 5(S)-(2′-hydroxyethoxy)-20(S)-camptothecin that was substantially free of 5(R)-(2′-hydroxyethoxy)-20(S)-camptothecin by repeatedly refluxing in n-butanol (generally 2-4 times; yield 25-35 grams).

5(R)-(2′-hydroxyethoxy)-20(S)-camptothecin was isolated from the mother liquor by dropwise addition of n-heptane followed by filtration using a 10.mu. Nutche filter.

5(RS)-(2′-hydroxyethoxy)-20(S)-camptothecin (diastereoisomeric mixture) was obtained as described in Example 26 of U.S. Pat. No. 6,177,439.

### Topoisomerase Assay:

Topoisomerase I introduces transient nicks in DNA at specific sites. Detection of these transient DNA nicks requires trapping the enzyme on DNA in a nicked intermediate complex using protein denaturants. The resulting covalent DNA/top I complexes contain nicked open circular DNA which can be detected by agarose gel electrophoresis (with ethidium bromide). Trapping nicked intermediates is relatively inefficient, however, inhibitors, such as the natural product camptothecin, stabilize the intermediate and lead to an increase in the nicked DNA product. This forms the basis for a mechanistic drug screen designed to allow detection of agents that affect topoisomerase I by stabilizing the cleaved intermediate complex.

The TopoGEN Topo I Drug Screening Kit (Topogen, Inc., Port Orange, Fla.) is designed to allow the investigator to quickly identify novel inhibitors of topoisomerase I. The kit allows the detection of novel compounds that either stabilize the nicked intermediate or otherwise inhibit catalytic activity of topoisomerase I.

Assay KIT used: Topogen Drug screening kit,
Manufacturer: TOPOGEN, Cat No: 1018.
Each reaction mix contains:

| | | |
|---|---|---|
| a. 10x Reaction buffer | 2 | μl |
| b. TOPO I enzyme | 2 | μl |
| c. pHOT I DNA | 1.2 | μl (0.5 ug) |
| d. Water | 14.8 | μl |
| e. Drug in DMSO | 1 | μl |
| Total | 20 | μl |

### Protocol

The above reaction mixture is incubated at 37 degree C. for 30 minutes. The reaction is terminated by adding 2 μl of 10% SDS and the mixture is vortexed rapidly (SDS should be added while at 37 degree C. as cooling the tubes might reseal the nicked DNA). 10× Dye, about 2.5 μl per tube, is added and equal volumes of a mixture of chloroform and isoamyl alcohol (24:1) is added and centrifuged at 13000 rpm for 10 minutes. Samples are loaded on a 1% agarose gel and electrophoresed for 1 hour at 80 volts. The gel was viewed on UV transilluminator and the densitometric estimation of the bands was calculated.

### Calculations

The density of the DNA bands of both super coiled and relaxed forms of DNA was measured using the densitometer. The band intensity of treated (with single concentration of the test drug) and without the drug (i.e., the Control) were recorded. The percentage of relaxed form DNA compared to the supercoiled DNA was calculated for all the lanes including treated and control.
% inhibition of Topoisomerase activity was calculated as:

=(100−(100×(1% inhibition in Control)×% inhibition in treated))

Table below shows the results of these tests and shows the in vitro topoisomerase I activities of 5(S)-(2′-hydroxyethoxy)-20(S)-camptothecin and 5(R)-(2′-hydroxyethoxy)-20(S)-camptothecin, which were substantially free of each other, compared with the activity of the racemic mixture 5(RS)-(2′-hydroxyethoxy)-20(S)-camptothecin.

TABLE
Topoisomerase I activity of 5(S)-(2′-hydroxyethoxy)-20(S)-
camptothecin, 5(R)-(2′-hydroxyethoxy)-20(S)-camptothecin
and 5(RS)-(2′-hydroxyethoxy)-20(S)-camptothecin.

| COMPOUND | IC50 (μM) |
|---|---|
| 5(S)-(2′-hydroxyethoxy)-20(S)- | 1.06 |
| camptothecin | |
| 5 (R)-(2′-hydroxyethoxy)-20(S)- | 22 |
| camptothecin | |
| 5 (RS)-(2′-hydroxyethoxy)-20(S)- | 12.5 |
| camptothecin | |

The results show that the 5(S)-(2′-hydroxyethoxy)-20(S)-camptothecin is about 21-fold more active than 5(R)-(2′-hydroxyethoxy)-20(S)-camptothecin and about 12 times more active than 5(RS)-(2′-hydroxyethoxy)-20(S)-camptothecin in inhibiting topoisomerase I. Such differences in activity would not be expected based on structural differences between the diastereomers since it is known that, particularly in view of the importance of the E-ring in enzyme activity.

### Example 22

This example shows the anti-tumor activity of 5(S)-(2′-hydroxyethoxy)-20(S)-camptothecin against NCl-H460 (human small cell lung carcinoma) xenografts in nude mice versus the activity of 5(RS)-(2′-hydroxyethoxy)-20(S)-camptothecin.

Samples of 5(S)-(2′-hydroxyethoxy)-20(S)-camptothecin and 5(RS)-(2′-hydroxyethoxy)-20(S)-camptothecin were provided as described in Example 21.

Protocol of Comparison Study of 5(RS)-(2′-hydroxyethoxy)-20(S)-camptothecin and 5(S)-(2′-hydroxyethoxy)-20(S)-camptothecin Against NCl-H460 Xenograft in Nude Mice

To perform the NCl-H460 xenograft study, NCl-H460 tumor pieces measuring .about.60 mm³ were implanted in the space of dorsal lateral flanks of female athymic nude mice to initiate tumor growth. When the tumors were grown to .about.150-1000 mm³, animals were randomized into groups of five prior to initiating therapy. Each gram of 5(RS)-(2′-hydroxyethoxy)-20(S)-camptothecin was formulated to contain 102.65 mg active compound, 801.62 mg hydroxylpropyl beta cyclodextran, 80.62 mg dextrose anhydrous and 13.33 mg sodium carbonate. Each gram of 5(S)-(2′-hydroxyethoxy)-20(S)-camptothecin was formulated to contain 105.57 mg active compound, 800.99 mg hydroxylpropyl beta cyclodextran, 80.13 mg dextrose anhydrous and 13.34 mg sodium carbonate. Each gram of placebo was formulated to contain 895.2 mg hydroxylpropyl beta cyclodextran, 89.52 mg dextrose anhydrous and 14.9 mg sodium carbonate. Each formulation was dissolved in 2 ml sterile water and administered through oral route in a (d×5)2 schedule. Tumor diameters were measured twice a week using a vernier caliper.

Tumor volumes were calculated assuming tumors to be ellipsoid using the formula: V=(D×d²)/2, where V (mm³) is tumor volume, D is longest diameter in mm and d is shortest diameter in mm. Change in tumor volumes (.DELTA.) for each treated (T) and control (C) group were calculated by subtracting the mean tumor volume on the first day of treatment (starting day) from the mean tumor volume on the specified observation day. These values were used to calculate a percentage growth (% T/C) using the formulas:

%T/C=(ΔT/ΔC)×100, where ΔT>0, or

%T/C=(ΔT/ΔTi)×100, where ΔT<0,

and Ti is the mean tumor volume.

Percentage tumor growth inhibition (% TGI) was then calculated using the formula: % TGI=100−% TC.

All of the mice bearing subcutaneous tumors measuring approximately 150-800 mm³ were treated with test compound through oral gavage using a (d×5)2 schedule. Tumor diameters were measured twice in a week using vernier calipers and tumor volumes were calculated assuming tumors to be ellipsoid using the formula V=(D×d²)/2 where V (mm³) is tumor volume, D is longest diameter in mm., and d is shortest diameter in mm. Changes in tumor Volumes (Δ volumes) for each treated (T) and control (C) group are calculated, by subtracting the mean tumor volume on the first day of treatment (starting day) from the mean tumor volume of on the specified observation day. These values are used to calculate a percentage growth (% T/C) using the formula:

%T/C=(ΔT/ΔC)×100, where ΔT>0; or =(ΔT/ΔTi)×100, where ΔT<0, where Ti is the mean tumor volume at the start of treatment.

Percentage tumor growth inhibition was calculated using the formula:

Percentage Tumor growth inhibition=100−% T/C.

Tumor regressions are defined as partial if the tumor volume decreases to 50% or less of the tumor volume at the start of the treatment without dropping below to 63 mm.sup.3. Complete regression is defined if the tumor volume drops to below measurable limits (<63 mm³).

The percentage body weight change in comparison to starting day body weight of each animal was calculated using the formula: Percentage Body weight change=[(Body weight on specified observation day-Body weight on starting day)/Body weight on starting day]×100.

The other parameter observed was mortality.

The results of the tests are shown in Table below, where the tumor growth inhibition and the mortality is shown for each of the two test compounds and for the control.

TABLE
Effect of 5(S)-(2′-hydroxyethoxy)-20(S)-camptothecin and 5(RS)-(2′-
hydroxyethoxy)-20(S)-camptothecin on tumor growth inhibition
and mortality of nude mice having NCI-H460 (human small cell lung
carcinoma) xenografts.

| | | % Tumor Growth | |
|---|---|---|---|
| Compound | Dose (mg/kg) | Inhibition | Mortality |
| 5(S)-(2′- | 2 | 68 | 0/5 |
| hydroxyethoxy)- | | | |
| 20(S)-camptothecin | | | |
| “—” | 4 | 76 | 0/5 |
| 5(RS)-(2′- | 2 | 60 | 0/5 |
| hydroxyethoxy)- | | | |
| 20(S)-camptothecin | | | |
| “—” | 4 | 64 | 0/5 |

The data from this test showed that 5(S)-(2′-hydroxyethoxy)-20(S)-camptothecin demonstrated better in vivo activity against NCl-H460 (human small cell lung carcinoma) xenografts in nude mice than the diastereoisomer 5(RS)-(2′-hydroxyethoxy)-20(S)-camptothecin. As shown in the Table, the administration of 5(S)-(2′-hydroxyethoxy)-20(S)-camptothecin led to unexpected increase in the inhibition of tumor growth in comparison with the administration of 5(RS)-(2′-hydroxyethoxy)-20(S)-camptothecin at identical doses (68% vs 60% at 2 mg/kg, and 76% vs 64% at 4 mg/kg) without an increase in mortality.

### Example 23

This example illustrates the efficacy of 5(S)-(2′-hydroxyethoxy)-20(S)-camptothecin versus 5(R)-(2′-hydroxyethoxy)-20(S)-camptothecin in inhibiting in vitro cell proliferation in a Sulphorhodamine B (SRB) assay.

Samples of 5(S)-(2′-hydroxyethoxy)-20(S)-camptothecin and 5(R)-(2′-hydroxyethoxy)-20(S)-camptothecin were provided as described in Example 21.

### Protocol for In Vitro Cell Growth Assay:

Cell proliferation was evaluated by Sulphorhodamine B (SRB) assay where the amount of dye bound to the cells after staining gives a measure of cell growth. Refer to: JNCI, vol 83, No. 11, Jun. 5, 1991, which is incorporated herein by reference.

Briefly, cells (34 human cancer cell lines represented by bladder, breast, CNS, colon, epidermoid, lung, ovarian, melanoma, prostate, renal and uterine cancers) were seeded on a 96-well cell culture plates at a concentration of 10,000 cells per well and incubated at 37 degree C. in a CO₂ incubator. Twenty-four hours later, cells were treated with different concentrations of andrographolide dissolved in DMSO to a final concentration of 0.05% in the culture medium and exposed for 48 h. Cells were fixed by adding ice-cold 50% trichloroacetic acid (TCA) and incubating for 1 h at 4.degree. C. The plates were washed with distilled water, air dried and stained with SRB solution (0.4% wt/vol in 1% Acetic acid) for 10 min at room temperature. Unbound SRB was removed by washing thoroughly with 1% acetic acid and the plates were air-dried. The bound SRB stain was solubilized with 10 mM Tris buffer, and the optical densities were read on a spectrophotometric plate reader at a single wavelength of 515 nm. At the time of drug addition separate reference plate for cell growth at time 0 h (the time at which drugs were added) was also terminated as described above. From the optical densities the percentage growths were calculated using the following formulae:

If T is greater than or equal to T₀,

percentage growth=100×[(T−T₀)/(C−T₀)]; and

if T is less than T₀,

percentage growth=100×[(T−T₀)/T₀)].

Where T is optical density of test, C is the optical density of control and T₀ is the optical density at time zero.

From the percentage growths a dose response curve was generated and GI₅₀ values were interpolated from the growth curves. Table below shows the results.

TABLE
GI50 values for 5(S)-(2′-hydroxyethoxy)-20(S)-camptothecin
versus 5(R)-(2′-hydroxyethoxy)-20(S)-camptothecin.

| Compound | GI₅₀ (μM) |
|---|---|
| 5(S)-(2′-hydroxyethoxy)-20(S)- | 5.0 |
| camptothecin | |
| 5(R)-(2′-hydroxyethoxy)-20(S)- | 14.6 |
| camptothecin | |

The results of this test showed that the 5(S)-(2′-hydroxyethoxy)-20(S)-camptothecin diastereoisomer was almost three times more active than the 5(R)-(2′-hydroxyethoxy)-20(S)-camptothecin diastereoisomer against cell proliferation in the test.

### Example 24

This example illustrates the efficacy of 5(S)-(2′-hydroxyethoxy)-20(S)-camptothecin in several osteosarcoma tumor models.

Samples of 5(S)-(2′-hydroxyethoxy)-20(S)-camptothecin and 5(RS)-(2′-hydroxyethoxy)-20(S)-camptothecin were provided as described in Example 21.

This test was carried out with the use of the methods described in Cancer, Res., Oct. 15, 64:20:7491-9 (2004), and in Clin. Cancer Res., Nov. 15:9 (15):5442-53 (2003).

All mice bearing subcutaneous (“Sc”) tumors measuring approximately 0.2-1 cm in diameter were treated with a test compound by oral gavage using [(d×5)2]3 schedule. Tumor diameters were measured every 7 days using Vernier calipers and tumor volumes were calculated, assuming tumors to be spherical, using the formula [π/6)×d³], where d is the mean diameter. The tumor response to the test compound was defined as follows: For individual tumors, partial regression (“PR”) was defined as a volume regression >50%, but with measurable tumor at all times. Complete regression (“CR”) was defined as disappearance of measurable tumor mass at some point within 12 weeks after initiation of therapy. Maintained CR is defined as no tumor re-growth within a 12-week study time frame. This time point was chosen because all studies lasted at least 12 weeks. Mice that died before the end of the 12-week study time, and prior to achieving a response, were considered as failures for tumor response. The results (dose of 28 mg/kg) are presented in Table below.

TABLE
Efficacy of 5(S)-(2′-hydroxyethoxy)-20(S)-camptothecin versus
5(RS)-(2′-hydroxyethoxy)-20(S)-camptothecin in mouse tumor
regression models.

| | 5(S)-(2′-hydroxyethoxy)- | 5(RS)-(2′-hydroxyethoxy)- |
|---|---|---|
| Xenograft | 20(S)-camptothecin | 20(S)-camptothecin |
| Osteosarcoma-29 | 6+ | 5+ |
| Osteosarcoma-17 | 6+ | 4+ |
| Osteosarcoma-2 | 6+ | 5+ |
| Osteosarcoma-32 | 6+ | 3+ |

6+: Maintained Complete Regression
5+: Complete Regression
4+: Partial Regression
3+: Stable Disease

As shown in Table, administration of 5(S)-(2′-hydroxyethoxy)-20(S)-camptothecin led to unexpectedly improved results in comparison with the administration of 5(RS)-(2′-hydroxyethoxy)-20(S)-camptothecin, as indicated by complete regression (6+) achieved with 5(S)-(2′-hydroxyethoxy)-20(S)-camptothecin in all four xenograft lines.

The data provided in Examples 23 and 24 illustrates that 5(S)-(2′-hydroxyethoxy)-20(S)-camptothecin has unexpectedly improved activity/potency profile in several test models. Furthermore, while 5(S)-(2′-hydroxyethoxy)-20(S)-camptothecin is substantially more potent than 5(R)-(2′-hydroxyethoxy)-20(S)-camptothecin, the increase in potency is not accompanied by a commensurate increase in toxicity.

### Example 25

This example shows the human bone marrow toxicity of 5(S)-(2′-hydroxyethoxy)-20(S)-camptothecin versus 5(R)-(2′-hydroxyethoxy)-20(S)-camptothecin.

Samples of 5(S)-(2′-hydroxyethoxy)-20(S)-camptothecin and 5(R)-(2′-hydroxyethoxy)-20(S)-camptothecin were provided as described in Example 21.

### Protocol for Human Bone Marrow Assay:

Methocult.TM. GF (Cat No: H4534, Poietics, Biowhittakar, USA) medium comprising Methycellulose in iscove's MDM, Fetal bovine serum, Bovine serum albumin, 2-Mercaptoethanol, L-Glutamine, rhStem cell factor, rhGM-CSF and rhIL-3 was used for the assay. Human bone marrow mononuclear cells (Cat No. 2M-125C, Poietics, Biowhittakar, USA) were mixed with Methocult GF and the cell density was adjusted to 3×10⁵ cells/ml. From this preparation, 500 μL aliquots were made and 2.5 μL of 20× drug solution or vehicle was added to each aliquot and mixed thoroughly. 100 μL of clonogenic medium was plated into each well and the plates were allowed to gel at 4.degree. C. for 15 minutes. Plates were incubated at 37 degree C. in a fully humidified atmosphere of 5% CO₂ in an incubator for 14 days. CFU-GM colonies were counted under an inverted microscope as aggregates of 50 cells or more. The percentage colony inhibition was calculated using the following formula: 100−[(number of colonies in drug treated wells/Number of colonies in control wells)×100].

The in vitro potency of the two diastereomers against cancer cell lines had been compared with their in vitro toxicity in healthy cells. Table below presents the results of the bone marrow toxicity comparison study in human cells.

TABLE
GI₉₀ values for 5(S)-(2′-hydroxyethoxy)-20(S)-camptothecin and
5(R)-(2′-hydroxyethoxy)-20(S)-camptothecin for human bone marrow
cells in vitro

| Compound | GI₉₀ (μL) |
|---|---|
| 5(S)-(2′-hydroxyethoxy)-20(S)- | 0.69 |
| camptothecin | |
| 5(R)-(2′-hydroxyethoxy)-20(S)- | 0.89 |
| camptothecin | |

With reference to the data shown in Tables above, it can be seen that while 5(S)-(2′-hydroxyethoxy)-20(S)-camptothecin is unexpectedly almost 3 times more potent against 34 human cancer cell lines (including bladder, breast, CNS, colon, epidermoid, lung, ovarian, melanoma, prostate, renal and uterine cancer cells) than 5(R)-(2′-hydroxyethoxy)-20(S)-camptothecin, the toxicities of both diastereomers are comparable. In fact, if the safety margin is estimated as the ratio of GI₉₀ for human cell toxicity to GI₉₀ for anticancer activity, as shown in Table above, it is apparent that 5(S)-(2′-hydroxyethoxy)-20(S)-camptothecin is unexpectedly superior to 5(R)-(2′-hydroxyethoxy)-20(S)-camptothecin and 5(RS)-(2′-hydroxyethoxy)-20(S)-camptothecin as a pharmaceutical compound for treatment of cancer. Thus, the 5(S)-(2′-hydroxyethoxy)-20(S)-camptothecin compound has increased efficacy with respect to treatment of cancer in comparison with the R-diastereomer and the mixture of diastereomers. In fact, it is unexpectedly important to minimize the amount of the 5(R)-(2′-hydroxyethoxy)-20(S)-camptothecin present in the 5(S)-(2′-hydroxyethoxy)-20(S)-camptothecin to be given to patients.

TABLE
Ratio of GI₉₀ for human cell toxicity to GI₅₀ for anticancer activity
for 5(S)-(2′-hydroxyethoxy)-20(S)-camptothecin and
5(R)-(2′-hydroxyethoxy)-20(S)-camptothecin.

| | Safety Margin |
|---|---|
| Compound | GI₉₀/GI₅₀ |
| 5(S)-(2′-hydroxyethoxy)-20(S)- | 0.14 |
| camptothecin | |
| 5(R)-(2′-hydroxyethoxy)-20(S)- | 0.06 |
| camptothecin | |

### Example 26

This example shows the effect of the presence of 5(R)-(2′-hydroxyethoxy)-20(S)-camptothecin on the bioavailability of 5(S)-(2′-hydroxyethoxy)-20(S)-camptothecin in rats and mice.

Samples of 5(S)-(2′-hydroxyethoxy)-20(S)-camptothecin (“5(S)-CPT”) and 5(RS)-(2′-hydroxyethoxy)-20(S)-camptothecin (“5(RS)-CPT”) were provided as described in Example 21.

### Bioavailability in Male Wistar Rats:

5(S)-CPT (2.5 mg/kg) and 5(RS)-CPT (5 mg/kg, including 2.5 mg/kg of 5(S)-CPT in the mixture) were been administered to male Wistar Rats to evaluate oral pharmacokinetics.

Male Wistar rats, 6-8 weeks of age and weighing between 205 and 218 g were divided into groups of four rats. The oral pharmacokinetics test was carried out in overnight fasted condition and intravenous pharmacokinetics was carried out in fed condition. The test drugs were administered as a solution by oral gavage or lateral tail vein injection. Sparse blood samples of about 250 microliters were collected from retro-orbital plexus at designated time points into microcentrifuge tubes containing 25 microliters of EDTA. Plasma was separated by centrifuging blood at 12,800 rpm for 2 min and refrigerated until analysis.

Samples were tested for the presence of the test drug as follows. An aliquot of 100 μL plasma (stored at 8.degree. C.) was precipitated with 400 μL of cold methanol for the estimation of total (lactone+carboxylate). Following mixing for 2 min. and centrifugation for 4 min. at 12,800 rpm, clear supernatant was separated into a 300.mu.l auto-sampler vial and 20 μL of this mixture was injected onto an analytical column for HPLC analysis. Concentrations of the test drug were calculated from the linearity plotted by spiking known concentrations of the test drug in blank rat plasma. The pharmacokinetics of the test drug was calculated using non-compartmental analysis.

The results of the study are presented in Table below

TABLE
Oral pharmacokinetic parameters of 5(S)-CPT in male Wistar rats.

| Compound | Dose | AUC(o-t) μM * h |
|---|---|---|
| 5(S)-CPT | 5 mg/kg | 5.76 |
| 5(RS)-CPT | 5 mg/kg | 5.08 |
| | (2.5 mg/kg 5(S)-CPT + | |
| | 2.5 mg/kg 5(R)-CPT) | |
| Contribution of 5(S)-CPT | 2.5 mg/kg | 1.21 |
| in 5(RS)-CPT | | |

### Bioavailability in Swiss Albino Mice:

5(S)-CPT (2.5 mg/kg) and 5(RS)-CPT (5 mg/kg, including 2.5 mg/kg of 5(S)-CPT in the mixture) were been administered to Swiss Albino mice to evaluate oral pharmacokinetics.

Swiss Albino mice, 3-6 weeks of age and weighing between 28-34 g were used in the study. Twelve mice were used per study. The oral pharmacokinetics test was carried out in overnight fasted condition and intravenous pharmacokinetics was carried out in fed condition. The test drugs were administered as a solution by oral gavage or lateral tail vein injection. Sparse blood samples of about 250 microliters were collected from retro-orbital plexus at designated time points into microcentrifuge tubes containing 25 microliters of EDTA. Plasma was separated by centrifuging blood at 12,800 rpm for 2 min and refrigerated until analysis.

Samples were tested for the presence of the test drug as follows. An aliquot of 100.mu.l plasma (stored at 8.degree. C.) was precipitated with 400.mu.l of cold methanol for the estimation of total (lactone+carboxylate). Following mixing for 2 min. and centrifugation for 4 min. at 12,800 rpm, clear supernatant was separated into a 300.mu.l auto-sampler vial and 20.mu.l of this mixture was injected onto an analytical column for HPLC analysis. Concentrations of the test drug were calculated from the linearity plotted by spiking known concentrations of the test drug in blank rat plasma. The pharmacokinetics of the test drug was calculated using non-compartmental analysis. The results of the study are presented in Table below.

TABLE
Oral pharmacokinetic parameters of 5(S)-CPT in Swiss Albino mice

| Compound | Dose | AUC(o-t) μM * h |
|---|---|---|
| 5(S)-CPT | 5 mg/kg | 5.18 |
| 5(RS)-CPT | 5 mg/kg | 5.20 |
| | (2.5 mg/kg 5(S)-CPT + | |
| | 2.5 mg/kg 5(R)-CPT) | |
| Contribution of 5(S)-CPT | 2.5 mg/kg | 1.1 |
| in 5(RS)-CPT | | |

With reference to Tables 7 and 8, the “Contribution of 5(S)-(2′-hydroxyethoxy)-20(S)-camptothecin” is the Area Under Curve (“AUG”) that can be attributed to the S-diastereomer in the RS diastereomeric mixture. As can be seen from Tables above, the presence of 5(R)-(2′-hydroxyethoxy)-20(S)-camptothecin unexpectedly decreases bioavailability of the desired 5(S) diastereomer. Moreover, it is believed that such unexpected decrease in bioavailability for the desired diastereomers would also be observed in human patients. On the basis of the above, the inventors have recognized that minimization of the amount of the R diastereomers impurity in 5(S)-(2′-hydroxyethoxy)-20(S)-camptothecin is desirable.

### Example 27

This example illustrates the efficacy of 5(S)-(2′-hydroxyethoxy)-20(S)-camptothecin versus 5(R)-(2′-hydroxyethoxy)-20(S)-camptothecin and 5(RS)-(2′-hydroxyethoxy)-20(S)-camptothecin against BCRP mutant and Breast cancer resistance protein (BCRP) over expressing Saos-2 cells.

Samples of 5(S)-(2′-hydroxyethoxy)-20(S)-camptothecin, 5(R)-(2′-hydroxyethoxy)-20(S)-camptothecin and 5(RS)-(2′-hydroxyethoxy)-20(S)-camptothecin were provided as described in Example 21.

### Protocol for Breast Cancer Resistance Protein (BCRP) Assay:

The anticancer effect of 5(S)-(2′-hydroxyethoxy)-20(S)-camptothecin & 5(R)-(2′-hydroxyethoxy)-20(S)-camptothecin were evaluated versus the racemate 5(RS)-(2′-hydroxyethoxy)-20(S)-camptothecin on Saos-2 cells over expressing functional BCRP#4 and non-functional BCRP mut#10. The human osteosarcoma cell line, Saos-2, was obtained from ATCC (American Type Culture Collection, Cat#HTB-85, Manassas, Va.) and were maintained in DMEM containing 10% fetal bovine serum, 1% penicillin/streptomycin, and 2 mM glutamine. Saos-2 cells were transfected with either BCRP#4 to over express functional BCRP or BCRP#10 to over express non-functional BCRP transporter. The cells were plated in 96-well plates at a density of 1000 cells per each well in a 0.1 ml of medium and allowed to attach overnight. The next morning the medium was gently aspirated and serial dilutions of the compounds to be tested were added. The cells were incubated at 37.degree. C. in a 5% CO.sub.2 incubator. After 6 days of exposure to the test drugs, 10.mu.l of Alamar blue was added aseptically to each well and the plates were returned to the incubator for 6 hr. The amount of the fluorescent dye produced was measured on a Cytofluor.RTM. 2300 (Millipore, Bedford, Mass.) using an excitation wavelength of 530 nm and emission wavelength of 590 nm. The relative fluorescence units obtained were used to calculate the percentage growth at each concentration in relation to the untreated control values. From the percentage growth values the IC.sub.50 (inhibitory concentration required to inhibit the cell growth by 50% compared to control cells growth) values were derived. The resulting IC₅₀ values are presented in Table below.

TABLE
IC₅₀ values for 5(S)-(2′-hydroxyethoxy)-20(S)-camptothecin, 5(R)-(2′-
hydroxyethoxy)-20(S)-camptothecin and 5(RS)-(2′-hydroxyethoxy)-
20(S)-camptothecin against BCRP mutant and Breast cancer resistance
protein (BCRP) over expressing Saos-2 cells.

| Drug | BCRP mut#10 (IC₅₀ (nM)) | BCRP mut#4 (IC₅₀ (nM)) |
|---|---|---|
| 5(RS)-CPT | 387 | 1256 |
| 5(S)-CPT | 213 | 788 |
| 5(R)-CPT | 1299 | >2000 |

As shown in the Table, 5(S)-(2′-hydroxyethoxy)-20(S)-camptothecin is superior to 5(R)-(2′-hydroxyethoxy)-20(S)-camptothecin and 5(RS)-(2′-hydroxyethoxy)-20(S)-camptothecin in terms of its cytotoxic activity on BCRP mutant as well as BCRP over expressing Saos-2 cells. These results indicate that the rank order of cytotoxicity on both BCRP mut#10 and BCRP#4 was 5(S)-(2′-hydroxyethoxy)-20(S)-camptothecin>5(RS)-(2′-hydroxyethoxy)-20-(S)-camptothecin>5(R)-(2′-hydroxyethoxy)-20(S)-camptothecin. 5(S)-(2′-hydroxyethoxy)-20(S)-camptothecin was .about.6 and >2.5 fold more cytotoxic than 5(R)-(2′-hydroxyethoxy)-20(S)-camptothecin on BCRP mut#10 and BCRP#4 over expressing Saos-2 cells, respectively.

We claim:

1. A powder composition for use in a pharmaceutical product, said composition comprising:
a) 5(S)-(2′-hydroxyethoxy)-20(S)-CPT; and
b) at least one cyclodextrin;
wherein said 5(S)-(2′-hydroxyethoxy)-20(S)-CPT includes less than 5% of 5(R)-(2′-hydroxyethoxy)-20(S)-CPT.

2. The powder composition of claim 1, wherein said 5(S)-(2′-hydroxyethoxy)-20(S)-CPT is substantially free from said 5(R)-(2′-hydroxyethoxy)-20(S)-CPT.

3. The powder composition of claim 1, wherein said 5(S)-(2′-hydroxyethoxy)-20(S)-CPT and said cyclodextrin are present in the form of an inclusion complex with one another.

4. The powder composition of claim 1, for which water solubility of said 5(S)-(2′-hydroxyethoxy)-20(S)-CPT is greater than 5 mg/ml.

5. The powder composition of claim 1, for which solubility of said 5(S)-(2′-hydroxyethoxy)-20(S)-CPT is greater than 25 mg/ml.

6. The powder composition of claim 1, which has residual moisture content ranging from about 2 to about 8 percent by weight.

7. The powder composition of claim 1, wherein said cyclodextrin is a hydrophilic cyclodextrin.

8. The powder composition of claim 7, wherein said cyclodextrin is hydroxypropyl betacyclodextrin.

9. The powder composition of claim 1, wherein said 5(S)-(2′-hydroxyethoxy)-20(S)-CPT and said cyclodextrin are present in the weight ratio ranging from about 1:1 to about 1:15.

10. The powder composition of claim 9, wherein said weight ratio is ranging from about 1:5 to about 1:10.

11. The powder composition of claim 3, further comprising at least one complexation enhancer.

12. The powder composition of claim 11, wherein said complexation enhancer includes a surfactant.

13. The powder composition of claim 11, wherein said surfactant is sodium lauryl sulphate.

14. The powder composition of claim 11, wherein said complexation enhancer includes an alkalizing agent.

15. The powder composition of claim 1, further comprising an alkalizing agent.

16. The powder composition of claim 14, wherein said alkalizing agent is an amino acid.

17. The powder composition of claim 15, wherein said amino acid is arginine, lysine or histidine.

18. The powder composition of claim 15, wherein said alkalizing agent is present in the amount of about 1 to 25% of the total weight of the powder composition.

19. The powder composition of claim 11, wherein said complexation enhancer comprises a combination of sodium lauryl sulphate and L-arginine.

20. The powder composition of claim 1, which produces a water solution with pH ranging from about 5 to about 9, measured upon dissolution of about 50 mg of the powder composition in about 1 ml of pure water.

21. The powder composition of claim 1, which is in the form of stabilized pharmaceutical formulation that possesses enhanced storage stability upon dissolution of the powder composition in an administration medium.

22. The powder composition of claim 21, which contains less than about 4% of total CPT-related impurities by total weight of the powder composition.

23. The powder composition of claim 21, which contains less than about 4% of any individual CPT-related impurity by total weight of the powder composition.

24. The powder composition of claim 23, which contains less than about 1% of any individual CPT impurity by total weight of the powder composition.

25. The powder composition of claim 22, 23, or 24, wherein said CPT-related impurity is a decarboxylated impurity of the chemical formula:

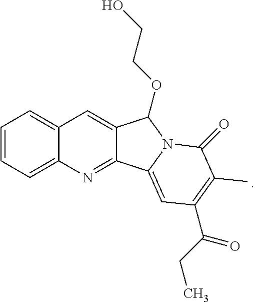

26. The powder composition of claim 22, 23, or 24, wherein said CPT-related impurity is a dimer impurity of the chemical formula

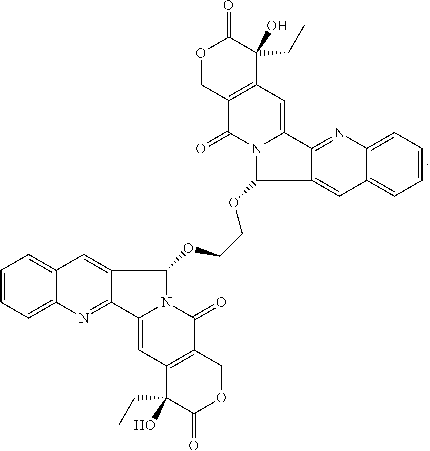

27. The powder composition of claim 22, 23, or 24, wherein said CPT-related impurity is a dehydro impurity of the chemical formula:

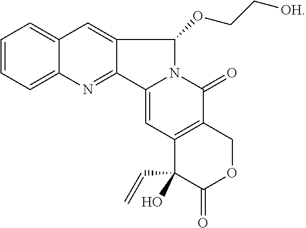

28. A pharmaceutical formulation for oral administration comprising a therapeutically effective dose of 5(S)-(2′-hydroxyethoxy)-20(S)-CPT in the form of the powder composition of claims 1 or 2.

29. The pharmaceutical formulation of claim 28, further comprising at least one pharmaceutically acceptable excipient.

30. The pharmaceutical formulation of claim 28, which releases 80% or more of said 5(S)-(2′-hydroxyethoxy)-20(S)-CPT into solution within 60 minutes after introduction of the pharmaceutical formulation into a biorelevant medium comprising 900 ml of 0.1 N hydrochloric acid at a temperature of 37° C.±0.5° C. in a USP Type II apparatus stirred at 75 rpm.

31. The pharmaceutical formulation of claim 30, which releases 80% or more of said 5(S)-(2′-hydroxyethoxy)-20(S)-CPT into solution within 30 minutes after introduction of the pharmaceutical formulation into the biorelevant medium.

32. The pharmaceutical formulation of claim 28, which comprises a capsule, said powder composition and said at least one excipient being filled into said capsule.

33. The pharmaceutical formulation of claim 28, which is a tablet.

34. The pharmaceutical formulation of claim 28, wherein said therapeutically effective dose is about 1 to about 100 mg.

35. The pharmaceutical formulation of claim 34, wherein said therapeutically effective dose is 5 mg.

36. The pharmaceutical formulation of claim 34, wherein said therapeutically effective dose is 10 mg.

37. The pharmaceutical formulation of claim 34, wherein said therapeutically effective dose is 25 mg.

38. The pharmaceutical formulation of claim 29, wherein said at least one pharmaceutically acceptable excipient is selected from the group consisting of diluents, disintegrants, glidants, and lubricants.

39. The pharmaceutical formulation of claim 32, wherein said capsule is size 00.

40. The pharmaceutical formulation of claim 32, wherein said capsule is size 3.

41. A pharmaceutical formulation for parenteral administration comprising i) a therapeutically effective dose of 5(S)-(2′-hydroxyethoxy)-20(S)-CPT in the form of the powder composition of claims 1 or 2; and ii) a container suitable for a parenteral pharmaceutical product.

42. The pharmaceutical formulation of claim 41, further comprising at least one parenterally-acceptable excipient.

43. The pharmaceutical formulation of claim 41, wherein said powder composition is in the form of a lyophilized powder.

44. The pharmaceutical formulation of claim 41, wherein said container is a vial, an ampoule or a syringe.

45. The pharmaceutical formulation of claim 41, wherein said therapeutically effective dose is from about 1 mg to about 100 mg.

46. The pharmaceutical formulation of claim 45, wherein said therapeutically effective dose is 5 mg.

47. The pharmaceutical formulation of claim 45, wherein said therapeutically effective dose is 25 mg.

48. The pharmaceutical formulation of claim 45, wherein said therapeutically effective dose is 50 mg.

49. A kit comprising a pharmaceutical formulation for parenteral administration, said kit comprising:
i) a therapeutically effective dose of 5(S)-(2′-hydroxyethoxy)-20(S)-CPT in the form of the powder composition of claim 1 or 2; and
ii) a pharmaceutically acceptable diluent for reconstitution.

50. The kit of claim 49, wherein the pharmaceutically acceptable diluent is sterile water for injection, dextrose solution, and/or saline solution.

51. A pharmaceutical formulation for parenteral administration comprising i) a therapeutically effective dose of 5(S)-(2′-hydroxyethoxy)-20(S)-CPT in the form of a sterile solution comprising a diluent suitable for a parenteral pharmaceutical product, a therapeutically effective dose of 5(S)-(2′-hydroxyethoxy)-20(S)-CPT, and a cyclodextrin, said 5(S)-(2′-hydroxyethoxy)-20(S)-CPT and said cyclodextrin being dissolved in said diluent; and ii) a container suitable for a parenteral pharmaceutical product;
wherein said formulation contains less than 5% of 5(R)-(2′-hydroxyethoxy)-20(S)-CPT with respect to total amount of 5(S)-(2′-hydroxyethoxy)-20(S)-CPT and 5(R)-(2′-hydroxyethoxy)-20(S)-CPT.

52. The pharmaceutical formulation of claim 51, wherein said 5(S)-(2′-hydroxyethoxy)-20(S)-CPT is substantially free from said 5(R)-(2′-hydroxyethoxy)-20(S)-CPT.

53. The pharmaceutical formulation of claim 51, wherein said cyclodextrin is hydroxypropyl betacyclodextrin.

54. The pharmaceutical formulation of claim 51, wherein said 5(S)-(2′-hydroxyethoxy)-20(S)-CPT and said cyclodextrin are present in the weight ratio ranging from about 1:1 to about 1:15.

55. The pharmaceutical formulation of claim 51, wherein said 5(S)-(2′-hydroxyethoxy)-20(S)-CPT is present at a concentration greater than 1 mg/ml.

56. The pharmaceutical formulation of claim 51, wherein said 5(S)-(2′-hydroxyethoxy)-20(S)-CPT is present at a concentration greater than 25 mg/ml.

57. The pharmaceutical formulation of claim 56, wherein said diluent is present at a volume smaller than an administration volume, said formulation being suitable for dilution with additional diluent.

58. The pharmaceutical composition of claim 51, further comprising at least one parenterally acceptable excipient.

59. The pharmaceutical composition of claim 58, wherein said at least one parenterally acceptable excipient is an osmolality adjustor.

60. The pharmaceutical composition of claim 59, wherein said osmolality adjustor is sodium chloride.

61. The pharmaceutical composition of claim 58, wherein said at least one parenterally acceptable excipient is a pH adjustor.

62. The pharmaceutical composition of claim 61, wherein said pH adjustor is an acetate, a citrate, or a phosphate.

63. The pharmaceutical composition of claim 58, wherein said at least one parenterally acceptable excipient is a preservative.

64. A method of making a powder composition that includes 5(S)-(2′-hydroxyethoxy)-20(S)-CPT and a cyclodextrin, said method comprising:
a) providing a solution or dispersion of 5(S)-(2′-hydroxyethoxy)-20(S)-CPT containing less than 5% of 5(R)-(2′-hydroxyethoxy)-20(S)-CPT and at least one cyclodextrin in a solvent;
b) combining said solution or dispersion with a complexation enhancer; and
c) removing said solvent;
thereby providing said powder composition.

65. The method of claim 64, wherein said 5(S)-(2′-hydroxyethoxy)-20(S)-CPT is substantially free from 5(R)-(2′-hydroxyethoxy)-20(S)-CPT.

66. The method of claim 64, wherein said step b) further comprises adding a bulking agent.

67. The method of claim 64, wherein said step c) comprises lyophilization.

68. The method of claim 64, wherein said step c) comprises spray-drying.

69. The method of claim 64, wherein said cyclodextrin is hydroxypropyl betacyclodextrin.

70. The method of claim 64, wherein said 5(S)-(2′-hydroxyethoxy)-20(S)-CPT and said cyclodextrin are present in the weight ratio ranging from about 1:1 to about 1:15.

71. The method of claim 70, wherein said weight ratio is ranging from about 1:5 to about 1:10.
